(19) United States
(12) Patent Application Publication
(10) Pub. No.: US 2004/0197317 A1
(43) Pub. Date: Oct. 7, 2004

(54) Persistent expression of candidate molecule in proliferating stem and progenitor cells for delivery of therapeutic products

(76) Inventors: Mahendra S. Rao, Timonium, MD (US); Mario R. Capecchi, Salt Lake City, UT (US)

Correspondence Address: TRASK BRITT, P.O. BOX 2550, SALT LAKE CITY, UT, 84110, US

(21) Appl. No.: 10/789,465

(22) Filed: Feb. 27, 2004

Related U.S. Application Data
(63) Continuation of application No. PCT/US04/00929, filed on Jan. 13, 2004.
(60) Provisional application No. 60/440,152, filed on Jan. 13, 2003.

(51) Int. Cl. A61K 48/00; C12N 15/85; C12N 5/08

(52) U.S. Cl. 424/93.21; 435/455; 435/366

(57) ABSTRACT

A method of obtaining and the resulting isolated progenitor or stem cell population of proliferating cells persistently expressing a candidate molecule. Further, novel methods of ex vivo gene product (e.g., protein) production and treating symptoms of neurological or neurodegenerative disorders are also provided.

### CROSS-REFERENCE TO RELATED APPLICATIONS

This application is a continuation of International Publication No. PCT/US04/00929, filed Jan. 13, 2003, which application claims the benefit. under 35 U.S.C. § 119(e) of U.S. Provisional Patent Application Serial No. 60/440,152 filed Jan. 13, 2003.

### TECHNICAL FIELD

The present invention relates generally to biotechnology and, particularly, to various methods of treating and using somatic stem cells and methods of delivering therapeutic products. More particularly, the present invention involves the use of homologous recombination in glial progenitor cells, mesenchymal stem cells, and astrocyte precursor cells, and includes the resulting cells.

### BACKGROUND

Delivery of therapeutic proteins for treatment of disease typically involves utilizing viral vectors as gene delivery vehicles. Therapeutic proteins may be produced by introducing exogenous DNA encoding the protein into appropriate cells. However, the use of viral vectors has limitations including the potential for generating replication-competent viruses during vector production. Similarly, recombination may occur between the introduced virus and endogenous retroviral genomes generating potentially infectious agents with novel cell specificities, host ranges, or increased virulence and cytotoxicity. The virus may also independently integrate into large numbers of cells and the limited cloning capacity in the retrovirus restricts therapeutic applicability. Further, there is a short lived in vivo expression of the product of interest. Thus, it can be appreciated that new methods of delivering therapeutic proteins that are independent from viral vectors would be useful.

Stem cells are self-renewing cells capable of generating daughter cells possessing self-renewal ability and differentiation potential properties similar to the parent stem cell. Certain stem cells such as hematopoietic stem cells have life-long self renewal ability while other stem cells have shorter self-renewal ability. Stem cells are classified based upon their tissue of origin and differentiation ability. Pluripotent embryonic stems cells (“ESCs”) can differentiate into any type of tissue. As ESCs differentiate, their lineage can be increasingly restricted into specific types of cells. For example, neural stem cells can generate derivatives in the central nervous system, while neural crest stem cells generate derivatives in the peripheral nervous system, liver stem cells, liver cells and pancreatic stem cells. Stem cells have been identified from multiple tissues including skin, blood, bone, gut and muscle and a partial list is provided in Table 1.

During differentiation, stem cells may generate more restricted precursors (also known as “progenitor” cells) which can undergo limited self-renewal but have a more restricted repertoire of differentiation. Glial progenitor cells, for example, can differentiate into multiple types of glial cells (i.e., astrocytes and oligodendrocytes) but not into neurons, while neuronal progenitors can generate multiple types of neurons but not astrocytes or oligodendrocytes. Restricted precursors have also been identified from multiple tissues and a partial list is provided in Table 2.

Stem and progenitor cells are being used in a variety of therapeutic paradigms including isolating cells from a purified or enriched mixture and either directly transplanting or transplanting the cells after a period in culture into a particular tissue or organ. In some cases, cells are transplanted after additional manipulations such as transfecting or infecting genes into cells, labeling cells with dyes or antibodies, or pre-treating cells with growth factors and cytokines.

Most methods of expressing genes in cells are limited because expression of the exogenous gene is down regulated or repressed by the cell's intrinsic mechanisms such as methylation, heterochromatin remodeling, and loss of stably expressing cells that are recognized as foreign. Evaluation of alternate methods to obtain stable expression in cells maintained for prolonged time periods is an ongoing research program in multiple laboratories (see Yanez, R J and Porter, A C, “Therapeutic Gene Targeting” Gene Ther. Feb. 5, 1998 (2): 149-159).

The possibility that homologous recombination could be used to insert genes into cells has been discussed and attempted off and on since the early 1980's. For example, Mario Capecchi et al. developed a method of selecting cells in which homologous recombination has occurred. See, e.g., U.S. Pat. Nos. 5,464,764, 5,487,992, 5,627,059, 5,631,153, and 6,204,061 (the contents of all of which are incorporated herein by this reference). However, success has been somewhat limited in stem cells because of inefficient gene targeting, the low natural abundance of stem cells in vivo, and the difficulty in maintaining stem cells or progenitor cells in an undifferentiated state in vitro for the number of cell divisions required to select a low efficiency homologous recombination event. Furthermore, somatic cell homologous recombination, including stem cells, has proven far more difficult.

To date, homologous recombination has been limited to embryonic stem cells for three primary reasons. First, initial efforts to use the technology for gene replacement in somatic cells (such as immortalized fibroblasts) were not encouraging; success was unacceptably infrequent and the failure of several influential laboratories discouraged serious attempts to adapt the Capecchi technology for somatic cells. It is likely that the efficiency of homologous recombination will prove to be highly cell type-specific.

Second, for most common uses of homologous recombination, somatic cells have substantial complications as compared to ESCs. Unlike ESCs, somatic cells require cell culture manipulations to ensure that both alleles of a given gene are replaced. Many reasons have been attributed to the difficulties with somatic cells including the inability to grow cells for long periods and the inability to select appropriate, efficient vectors. Thus, for the best appreciated uses of homologous recombination, the procedure in somatic cells is intrinsically more difficult and substantially more involved than for ESCs.

Third, under the best conditions, homologous recombination in mammalian ESCs occurs at a frequency of roughly one per million of the starting cell population. If the homologous recombination procedure is to be successfully adapted for use in any specific primary cell type, then the cell type should be amenable to at least 24 rounds of cell division in culture to yield roughly 10 million cells. For the best characterized hematopoietic stem cell type from bone marrow, no more than 2-3 cell divisions have been achieved in culture. However, ESCs are not ideal therapeutic candidates because they are derived from embryos which raise political and ethical considerations. Furthermore, ESCs may proliferate spontaneously to form tumors and may not respond appropriately to in vivo differentiation signals.

Thus, it can be appreciated that a need exists to identify a strategy to obtain persistent expression of candidate molecules in cells other than ESCs.

### DISCLOSURE OF THE INVENTION

The present invention involves a novel method of stable expression of molecules in stem or progenitor cells using a technique of homologous recombination in somatic cells. Somatic or progenitor cells may be grown in culture such that the somatic or progenitor cells remain undifferentiated, express TERT, maintain telmorase activity and demonstrate a capacity for self-renewal. In an embodiment, the stem or progenitor cells may comprise glial progenitor cells, mesenchymal stem cells, astrocyte precursor cells, and any mixtures thereof.

A gene of interest may be cloned into a construct or vector backbone such that expression of the protein of interest may be regulated by a constitutively active ubiquitous or cell type-specific promoter. The vector may be inserted into cultured stem or progenitor cells by a variety of methods, including, but not limited to electroporation, Lipofection™, cell fusion, retroviral infection, cationic agent transfer, CaPO₄, transfection and combinations thereof. The vector design may be such that it contains regions of homology with specific sequences in the human, rat or mouse genome. In an embodiment, the regions of homology may have 100% homology. Such homologous sequences may include but are not limited to the Rosa locus, the RNApoIII locus and the beta-actin locus. These homologous sequences allow recombination to occur between the inserted DNA and the homologous sequences in chromosomal DNA as the cell undergoes replication. The invention also includes a somatic or progenitor cell produced by this method.

The invention also includes stem or progenitor cells having DNA inserted into the homologous site that may be isolated and selected using a selectable gene marker. The cells may then be used for subsequent experiments including, but not limited to, transplanting the stem or progenitor cells into a subject such that replacement of a gene product corrects an abnormality or deficit. Examples of such abnormalities or defects include loss of a catalytic enzyme, reduction in levels of growth factors or their receptots, and novel expression of a protein in a-cell not normally expressing the protein.

Another embodiment of the invention includes generating stem or progenitor cell lines in which at least one homologous recombination event has successfully occurred such that at least one sequence has been placed at a selected site in the genome of the stem or progenitor cell such that the same selected site may be repeatedly targeted. For example, a first homologous recombination event may insert a gene sequence that enhances later homologous recombination events at the same location. The inserted gene sequence may be replaced with a third gene or fourth gene in a reproducible manner.

Yet another embodiment of the invention includes undertaking homologous recombination in a somatic cell and obtaining multiple clones of cells that express different candidate growth factors for evaluating the efficacy of growth factor delivery in vivo and allowing direct comparisons of gene expression.

Another embodiment of the invention includes undertaking homologous recombination in a particular locus and then reselecting the obtained clone for a second recombination event which duplicates the change introduced by the first recombination event at the second allele. Such homozygous mutant cells may be obtained by either reselecting using a higher concentration of the selection agent or undertaking a second recombination process as the first in the same cell line. Another embodiment includes modifying a promoter capable of controlling expression of the gene of interest. The modification may include replacing at least a portion of the promoter with a product capable of providing additional regulation of expression of the gene product.

A subject may be incapable of producing the gene of interest or may be incapable of expressing normal levels of a gene of interest. After homologous recombination has occurred, the gene of interest may be delivered to a subject using a purified or enriched population of the somatic or progenitor cells. Delivery may comprise in vitro or in vivo delivery of the gene of interest. In an embodiment, delivery may comprise expressing the gene of interest in the subject.

Another embodiment of the present invention includes an isolated population of glial progenitor cells capable of expressing an endogenous protein introduced into the glial progenitor cell through homologous recombination. The glial progenitor cell may lack MHC expression. The glial progenitor cells may be capable of differentiating, expressing TERT, maintaining telomerase activity and self-renewal.

Another embodiment of the present invention includes an isolated population of mesenchymal stem cells capable of expressing an endogenous protein introduced into the mesenchymal stem cell through homologous recombination. The mesenchymal stem cell may lack MHC expression. The mesenchymal stem cells may be capable of differentiating, expressing TERT, maintaining telomerase activity and self-renewal.

Another embodiment of the present invention includes an isolated population of astrocyte precursor cells capable of expressing an endogenous protein introduced into the astrocyte precursor cell through homologous recombination. The astrocyte precursor cells cell may lack MHC expression. The astrocyte precursor cells may be capable of differentiating, expressing TERT, maintaining telomerase activity and self-renewal. The invention also includes homologously recombined somatic stem or progenitor cells for use in treating disorders, including neurological or neurodegenerative disorders.

The invention also includes a method of gene therapy including using an isolated population of glial progenitor cells, mesenchymal stem cells, astrocyte precursor cells, or a mixture thereof, that express an endogenous protein introduced into the cells through homologous recombination for ex vivo gene therapy.

A method of manufacturing a pharmaceutical preparation for the treatment of a neurological or neurodegenerative disorder, comprising using the homologously recombined somatic stem or progenitor cells of the present invention, together with a pharmaceutically acceptable excipient.

### BRIEF DESCRIPTION OF THE DRAWINGS

FIG. 1 depicts examples of commercially available plasmids for homologous recombination in somatic cells. Note multiple promoters may be used and the backbone containing the targeting construct may vary. Plasmids may be transferred to the somatic cell by electroporation, Lipofection™, calcium phosphate mediated DNA transfer or by retroviruses.

FIG. 2 depicts examples of vectors that may be used according to the present invention. Vectors may be designed to utilize endogenous promoters, provide ectopic promoters or identify endogenous promoters.

FIG. 3 depicts an example of recombination where the replaced gene utilizes the endogenous promoter sequence to drive cell-type specific expression.

FIG. 4 depicts an example using a vector containing an IRES site to direct expression of a transcript from an endogenous promoter.

FIG. 5 depicts utilization of SA sites to disrupt the endogenous gene and generate a desired transcript or a fused transcript.

FIG. 6 is an example of cell type specific expression with Cre mediated recombination to remove the flanking selection sequences. Note that other systems including ΦC31/AttP/AttB or Flγ/FRT may also be used.

FIG. 7 illustrates an example of repeated homologous recombination. Note repeat targeting may be performed in several manners and one example using Floxed sites is shown.

FIG. 8 illustrates glial progenitor stem cells (“GRP”) cells expressing telomerase activity (part A). NEP cells and E14 mixed cells were obtained from freshly dissected E10.5 and E14 embryos. A2B5 positive GRP cells were selected from E14 mixed cells sorted by flow cytometry. Extracts, equivalent to 1000 cells were analyzed for telomerase activity with standard TRAP assay. Levels were quantified and are presented in a table format (part b). “HI” samples are heat-inactivated controls. TERT expression was assessed by RT-PCR using gene specific primers (part c).

FIG. 9 illustrates immortalization of A2B5-immunoreactive cells. A2B5-immunoreactive cells were purified by immunopanning and immortalized using v-myc as an immortalizing oncogene. Some cells were grown in the presence of tetracycline and their proliferation rate assessed by BRDU incorporation (part C and part D), while other immortalized cells were cultured for 1 week in the presence of PDGF/T3, FBS, or CNTF to promote oligodendrocyte (part E) and astrocyte (parts F and G) differentiation. Part A shows a representative field stained with A2B5 (red) and DAPI (blue) to show that the isolates comprise of A2B5-immunoreactive cells (_{—}95%). Part B outlines the procedure followed to obtain immortalized subclones. Parts C and D show that the rate of proliferation as assessed by BRDU incorporation (red) is dependent on tetracycline, indicating that the tetracycline-regulatable v-myc is functional. Parts E, F, and G show that immortalized cells can differentiate in oligodendrocytes (Part E: Gal-C, red), A2B5_astrocytes (Part F: GFAP, green; A2B5, red) and A2B5_astrocytes (Part G: GFAP, green; A2B5, red). Note the difference in morphology of the A2B5_and A2B5_astrocyte populations (compare parts F and G).

FIG. 10 depicts characteristics of the immortalized cells. A2B5-immortalized cells were passaged (P7) and grown in DMEM/F12 medium supplemented with FGF (10 ng/ml). Cells were harvested after 510 days in culture as expression of different markers was tested by RT-PCR (parts A and B) or by immunocytochemistry (parts D-J). For some experiments, immortalized cells were differentiated and the acquisition of markers was assessed (parts C and H) and in other experiments, expression was compared with expression in non-immortalized cells (part J). Immortalized cells do not express PDGF-R, NF, or olig-2 (parts A and B) and only a subset of cells express Nkx2.2 (part G: Nkx2.2, red; A2B5, green) or GD-3 (part I: GD-3, red; DAPI, blue), which is similar to GD-3 expression seen in unimmortalized cells at E14.0 (part J: GD-3, red; A2B5, green). Most immortalized cells express nestin (part A; compare parts D and D_{—}, 4D4 (part F), and HNK-1 (part E). Expression of other glial precursor markers such as Ngn3, olig-1, and PLP/DM20 can also been seen. Note that only the DM20 splice form of the PLP/DM20 transcript can be detected by PCR (part A) and only more mature appearing cells are immunoreactive with PLP/DM20 antibody in the differentiated state.

FIG. 11 illustrates GFP-labeled subclones can differentiate into astrocytes and oligodendrocytes. A2B5-immortalized cells expressing GFP were isolated as described and passaged (P10) cells were grown in DMEM/F12 medium supplemented with FGF (10 ng/ml) and cells were harvested after 5-10 days in culture (parts B and B_) and integration of v-myc was assessed by Southern blot hybridization (part A). Cells were replated in conditions that promote astrocyte differentiation [parts C and C_; DMEM/F12, FGF (10 ng/ml), and BMP (10 ng/ml)] or oligodendrocyte differentiation [parts D and D_; DMEM/FI2, FGF (10 ng/ml), and growth factors]. GFP expressing cells show a single integration site using three different restriction enzymes (part A) and virtually all. GFP-expressing cells continue to express A2B5 under proliferation conditions (parts B and B_). GFP-expressing cells can differentiate into astrocytes (parts C and C_) or oligodendrocytes (parts D and D_) under appropriate growth conditions, indicating that expression of GFP does not alter the ability of this clone to differentiate into astrocytes and oligodendrocytes.

FIG. 12 illustrates an example of repeated homologous recombination. Note repeat targeting may be performed in several ways and one example using a single Floxed site is shown.

FIG. 13 depicts neomycin sensitivity in GRPs. Part A shows GRPs exponentially growing under high magnification. Part B shows GRPs plated without neomycin (G418) under low magnification. Part C shows GRPs plated with neomycin (G418) under low magnification.

FIG. 14 shows stable transfection of GRPs. Part A shows untransfected GRPs. Parts B and C show neomycin (G418) resistance clones.

FIG. 15 illustrates vector used in an embodiment of the presently claimed invention wherein IRES-neo sequences were cloned into the 3′ non-coding sequence (flanking exon 28) of the mouse Polr2a locus.

FIG. 16 illustrates targeted transgene integration by homologous recombination in mouse glial progenitor cells.

FIG. 17 depicts the PCR results from one embodiment of the presently claimed invention. Part A depicts PCR with oligonucleotides flanking presumptive IRES-neo insertion. Two clones (2 and 13) showed bands larger than wild-type. In part B, an additional PCR was performed with one oligonucleotide primer within IRES-neo and one in Polr2a sequence flanking the target vector.

### BEST MODE OF THE INVENTION

Homologous recombination has been used to create transgenic mice and to target in some loci in cell lines and some somatic cells. However, success has been variable and dependent upon developing appropriate conditions and vectors for a specific cell type. In general, cells must undergo sufficient number of cell divisions, be capable of being selected and of growing at low density to be viable candidates for homologous recombination. Few cells fulfill these criteria and consequently successful homologous recombination has been restricted to embryonic stem cells, immortalized cell lines and fibroblast cells.

As stated, ESC are not ideal therapeutic candidates in part because they may not respond appropriately to differentiation signals. However, intermediate-lineage glial progenitors have a differentiation repertoire restricted to forming glial tissue and are normally present in the adult brain and spine where they respond to in vivo signals. Further, the oligodendrocyte subtype is primarily responsible for producing myelin, the protective sheath surrounding nerve fibers in the central nervous system (“CNS”). Loss of oligodendrocyte cell function plays a major role in the onset of demyelinating disorders such as multiple sclerosis.

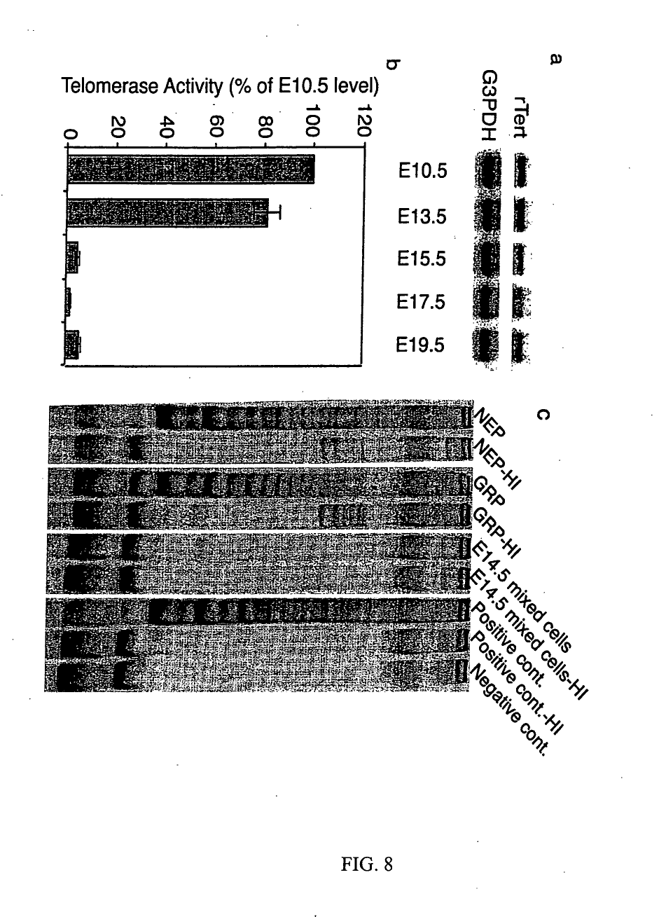

Methods of isolating purified populations of glial restricted precursor cells have been shown and offer the possibility of resolving the traditional obstacles to homologous recombination in somatic cells because it has been shown that glial progenitor cells may be maintained in culture for prolonged periods of time while retaining their characteristics. Further, it was recently demonstrated that glial progenitor cells may be immortalized, foreign genes may be introduced and the cells may be selected for expression of the foreign gene. See, Wu et al. “Isolation of a Glial-Restricted Tripotential Cell Line from Embryonic Spinal Cord Cultures” GLIA 38: 65-69 (2002) the contents of which are incorporated herein by reference. Further, glial progenitor cells express high telomerase levels. See, Sedivy, “Can Ends Justify the Means? Telomeres and the Mechanisms of Replicative Senescence and Immortalization in Mammalian Cells” PNAS USA 95: 9078-9081 (August 1998) the contents of which are incorporated herein by reference. According to the present invention, progenitor cells which are self-renewing for at least 20 passages, capable of differentiating into glial cells and telomerase positive are candidates for homologous recombination events. (See, FIG. 8.)

More than 90% of the CNS cells are glia and glial cell therapies are potentially important in the treatment of a wide range of neurological disorders including demyelinating and neurodegenerative disorders. Glial cells are essential for maintaining neuronal survival and normal function, modulating neurotransmitter metabolism, and synthesizing myelin to maintain optimal signal propagation between neurons. Loss of glial function plays a primary role in demyelinating disorders ranging from multiple sclerosis, spinal cord injury, subcortical stroke, cerebral palsy, and inherited disorders including leukodystrophies. Glial dysfunction is also a major factor in neurodegenerative diseases including Parkinson's disease, Amyotrophic Lateral Sclerosis (“ALS”), Huntington's disease and lysosomal storage disorders including, but not limited to, Tay-Sachs disease, Hurler syndrome, Gaucher's disease, Fabry's disease and Late Infantile Neuronal Ceroid Lipofuscinosis (“LINCL”). Thus, glial progenitor cells are an ideal therapeutic candidate.

The glial progenitor cells are also ideal therapeutic delivery vehicles because of their exceptional capacity to multiply, migrate to the site of infection and differentiate into oligodendrocyte and astrocyte subtypes. It is contemplated that such diseases may be treated in a variety of manners including genetically encoding glial progenitor cells to express exogenous protein factors and delivering the cells to damaged tissues, mobilizing endogenous progenitor stems cells by delivering inductive growth factors and/or cell replacement therapy. However, a major impediment to such therapies has been the lack of a suitable therapeutic candidate. The present invention provides a method of using homologous recombination to create viable therapeutic candidates.

One key to successful homologous recombination in stem or self-renewing progenitor cells (primary cells) is achieving the ability to propagate these cells essentially unchanged in culture for many generations. This may be accomplished directly by actually passaging the cells in culture for many generations or inferred from high expression levels of the enzyme telomerase that marks immortal cells. It was recently shown that glial progenitor cells may be maintained through more than 30 generations in culture as well as express high levels of telomerase, a biochemical marker for cell immortality. Mesenchymnal cells may also be propagated indefinitely in culture (more than 40 generations) and exhibit high telomerase levels. Other classes of stem and progenitor cells are expected to exhibit similar characteristics including, but not limited to astrocyte precursor cells. See, Sommer and Rao, “Neural Stem Cells and Regulation of Cell Number,” Progress in Neurobiology, 66: 1-18 (2002).

Initial failures with homologous recombination in somatic cells may be attributed to a lack of appreciation for the importance of critical experimental parameters. For example, a 100 percent match may be required between experimentally manipulated targeting sequences and target sequences in the cell. (See Yanez, R J and Porter, A C, “Therapeutic Gene Targeting” Gene Ther. Feb. 5, 1998 (2): 149-159.) The present invention demonstrates that homologous recombination occurs efficiently in at least one specific genetic locus in glial progenitor cells, mesenchymal stem cells, and astrocyte precursor cells.

The use of homologous recombination directed transgene integration for controlled drug delivery has been essentially ignored in the largely non-overlapping fields of stem cell research and homologous recombination. The present invention provides new characterization of the growth properties of stem and progenitor cell populations in culture and the technique of homologous recombination to define an unprecedented strategy to obtain persistent expression of candidate molecule in proliferating stem and progenitor cells.

In general, the homologous recombination process may be characterized as beginning with a cell into which DNA of interest is introduced. In the present invention, the starting cell may be any self-renewing somatic stem cell that differentiates into a glial cell type and is telomerase positive. Exemplary cells include but are not limited to glial progenitor cells, mesenchymal stem cells, and astrocyte precursor cells. Based upon the data obtained from the Examples herein, it is expected that homologous recombination according to the present invention will be possible in all progenitor cells having self-renewal ability, expressing telomerase, and having the ability to differentiate into glial cells.

After introduction of the DNA, homologous recombination is permitted to occur between the DNA of the cell and the introduced DNA such that the cell may then express a product encoded by the inserted DNA. In the present invention, DNA may be introduced into a particular locus in the DNA of the cell which is expressed in the progenitor cell or its differentiated progenitor. Examples of such loci include, but are not limited to Rosa locus, RNA pol II and genes specific to the progenitor cell type, for example, but not limited to cyclic nucleotide diphosphatase (“CNP”), myelin basic proteins (“MBP”) and proteolipid proteins (“PLP”).

According to the invention, DNA may be introduced into the cell by a variety of methods including, but not limited to electroporation, Lipofection™, cell fusion, retroviral infection, cationic agent transfer, CaPO₄, transfection and combinations thereof. The DNA to be introduced into the cell may be introduced in a variety of formats including, but not limited to, DNA constructs, DNA plasmids, lambda phage, BAC (bacterial artificial chromosome), and YAC (yeast artificial chromosome).

A homologously recombined stem or progenitor cell may be combined with a pharmaceutically acceptable carrier or excipient as known in the art. Suitable pharmaceutical carriers include inert solid diluents or fillers, sterile aqueous solutions and various organic solvents. The pharmaceutical compositions formed by combining a homologously recombined stem or progenitor cell and a pharmaceutically acceptable carrier may be administered in a variety of dosage forms such as tablets, powders, lozenges, syrups, injectable solutions and the like. Dosage may be made by a person of ordinary skill taking into account known considerations such as the weight, age, and condition of the subject being treated, the severity of the affliction, and the particular route of administration chosen.

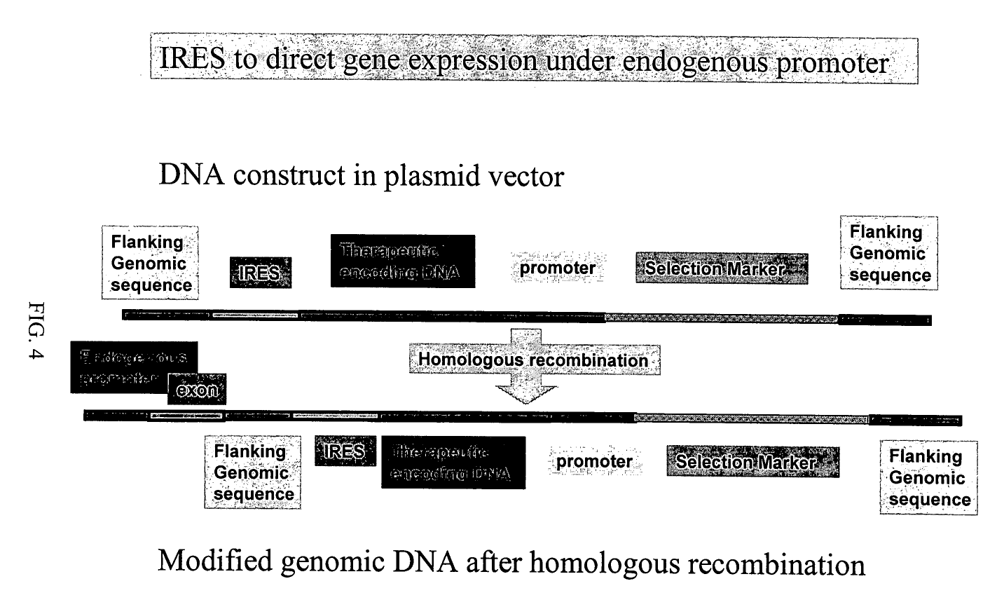

In a particular embodiment, an internal ribosome entry site (“IRES”) protein is inserted at a particular locus where homologous recombination will occur so that the recombined gene will be regulated by the endogenous promoter. (FIG. 4.)

Homologous recombination may also be employed to replace or modify a promoter for a gene of interest in a cell. Such a homologous recombination event may, for example, allow inducible control of the gene of interest. Vectors traditionally used in homologous recombination in embryonic stem cells may be used in the somatic stem cells. Examples of genes of interest include, but are not limited to, platelet derived growth factor (PDGF), epidermal growth factor (EGF), fibroblast growth factor (FGF), brain derived neurotrophic factor (BDNF), glial derived neurotrophic factor (GDNF) and ciliary neurotrophic factor (CNTF).

The present invention may be further understood by the following non-limiting examples.

### EXAMPLE I

To test the ability of glial progenitor cells to grow in culture, levels of telomerase activity, the ability to divide for prolonged periods in culture and the ability to deliver DNA into the cells using electroporation, Lipofection™ and retroviral infection were evaluated. See, Rao et al., “A Tripotential Glial Precursor Cell is Present in the Developing Spinal Cord” PNAS USA 95:3966-4001 (March 1998); Rao and Mayer-Proschel, “Glial-Restricted Precursors are Derived from Multipotent Neuroepithelial Stem Cells”. Developmental Biology 188: 48-63 (1997); U.S. Pat. No. 6,361,996 and U.S. Pat. No. 6,235,527, the contents of all of which are incorporated herein by reference.

FIG. 8 illustrates GRP cells expressing telomerase activity. NEP cells and E14 mixed cells were obtained from freshly dissected E10.5 and E14 embryos. A2B5 positive GRP cells were selected from E14 mixed cells sorted by flow cytometry. Extracts, equivalent to 1000 cells were analyzed for telomerase activity with standard TRAP assay. Levels were quantified and are presented in a table format (FIG. 8, part b). “HI” samples are heat-inactivated controls. TERT expression was assessed by RT-PCR using gene specific primers (FIG. 8, part c). Thus, glial progenitor cells are candidates for homologous recombination events.

### EXAMPLE II

A vector is designed for homologous recombination and it is shown that recombination may be achieved at a particular site using the designed vector.; A gene of interest is cloned into a vector backbone such that expression of the protein is regulated by a constitutively active ubiquitous or cell type-specific promoter. The vector is inserted into cultured progenitor cells by, for example, electroporation, Lipofection™ and/or cell fusion. The vector design is such that it contains regions of homology with specific sequences in the particular subject (e.g., human, rat or mouse) genome. Such homologous sequences include but are not limited to the Rosa locus, the RNApoIII locus and the beta-actin locus. These homologous sequences allow recombination to occur between the inserted DNA and the homologous sequences in chromosomal DNA as the cell undergoes replication.

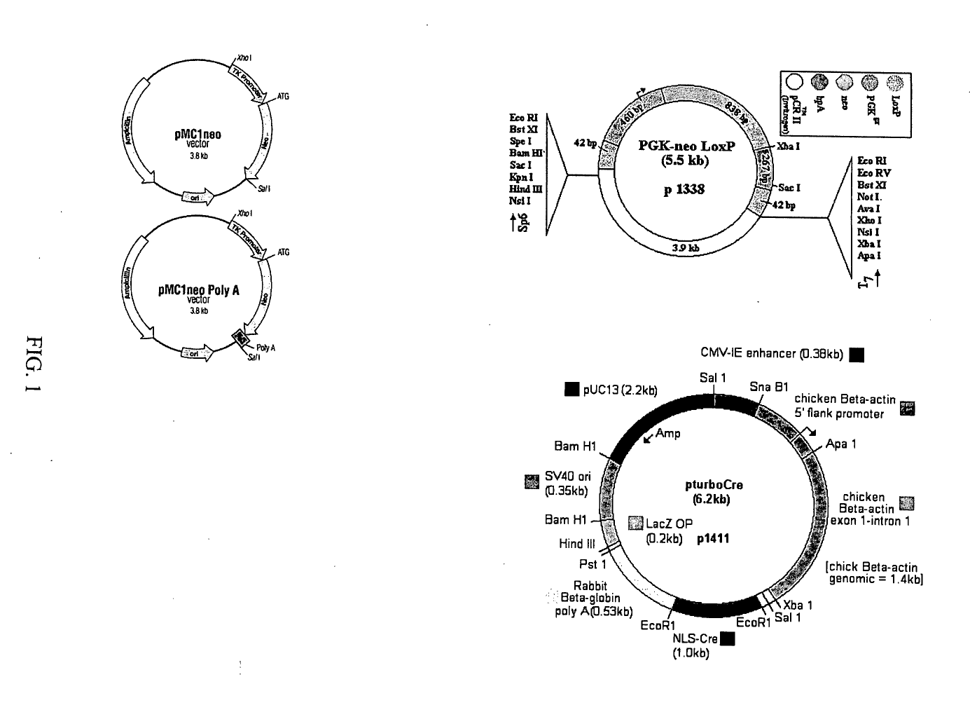

Site-specific integration requires the ability to obtain sufficient numbers of cells that can be grown in culture for a sufficient time period to successively select the cell in which a site specific reombination event has occurred. We have shown that for glial progenitor cells, astrocyte precursor cells, and mesenchymal stem cells, we can obtain cells in large numbers that self-renew, allow transfected genes to be expressed, and are amenable to selection using neomycin and puromycin. FIG. 1 depicts examples of prototype vectors which illustrates that electroporation may be used to insert DNA into cells. Tested methods of insertion of DNA include electroporation, Lipofection™, viral transfer, and calcium phosphate mediated transfer which suggests that any other standard commercially available gene delivery agent having an efficiency of at least 20% may be used according to the present invention.

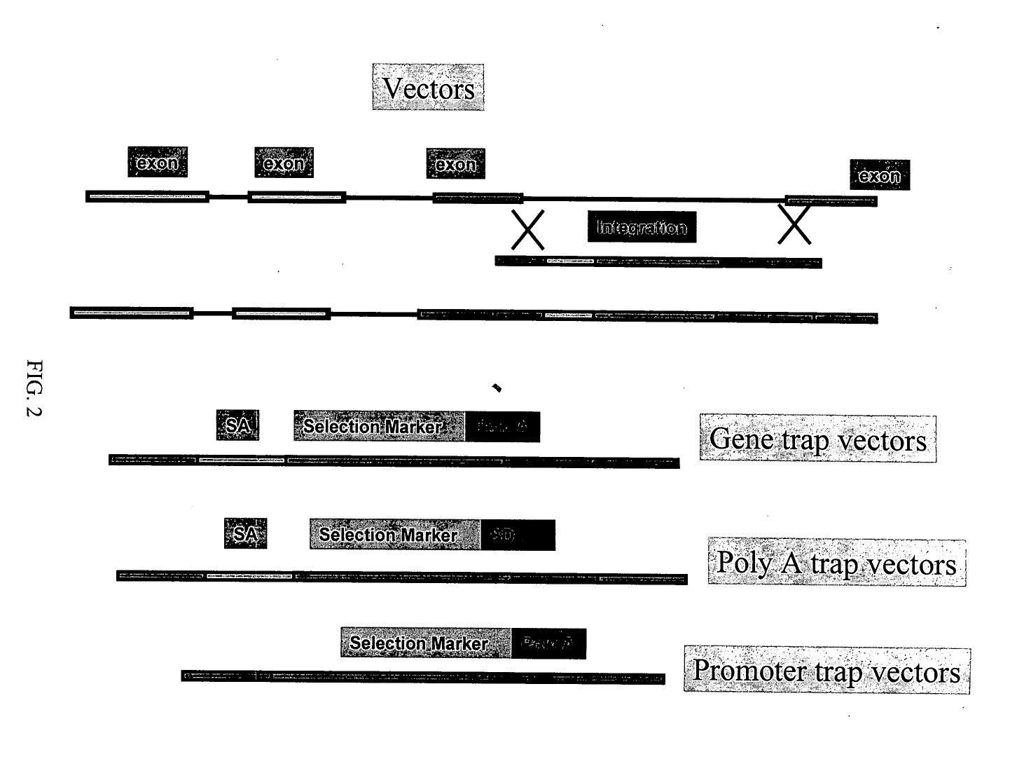

Constructs to target the Rosa 26 locus, RNA pol II and GAPDH loci have been developed to show that any cloned loci of interest may be targeted. Several variations of such plasmids have been used. Either promoter-containing or promoter-less constructs with or without splice donor or acceptor sites may be used. Constructs with IRES sites or floxed gene products may be made using methods that are well described and readily obtainable by one skilled in the art. A. detailed review of vectors and constructs used for homologous recombination is described in (Court et al., 2002, Copeland et al., 2001) and examples of some variants of vectors are described herein. (FIG. 2.)

A vector may be promoter-less without an enhancer to be integrated downstream of an endogenous enhancer (e.g., Rosa 26). According to the present invention, the vector may be a construct with an additional enhancer element that allows exogenous control of gene expression in addition to that provided by an endogenous enhancer as in the promoter-less vector. Promoters including, but not limited to CMV, PGK, prion proteins or any promoter suitable for driving expression in progenitor cell populations, may be integrated upstream of an endogenous gene, for example, one encoding GDNF.

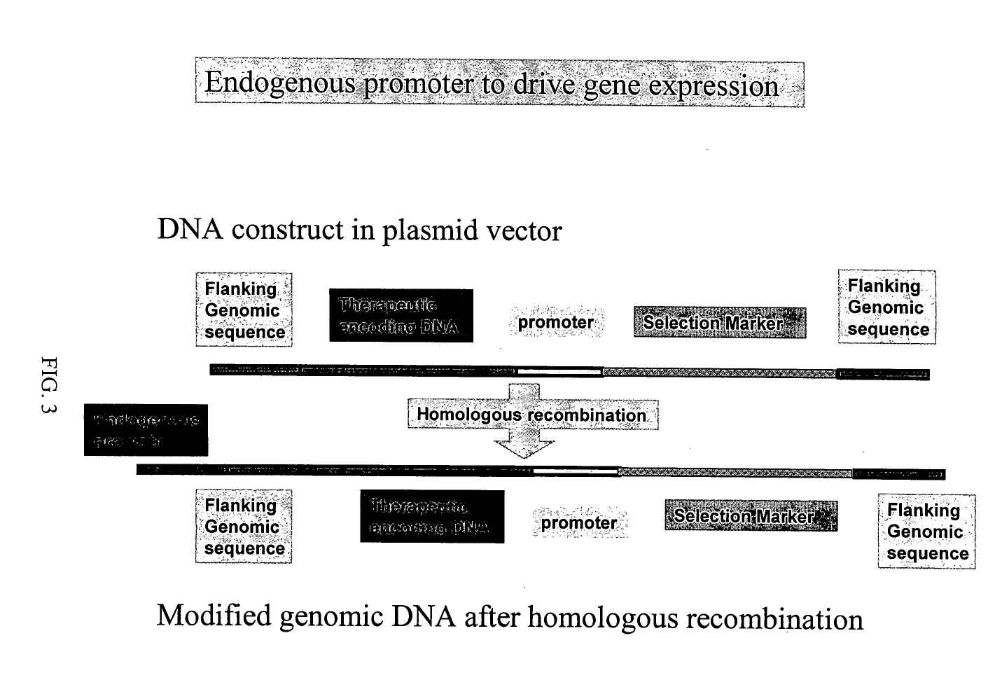

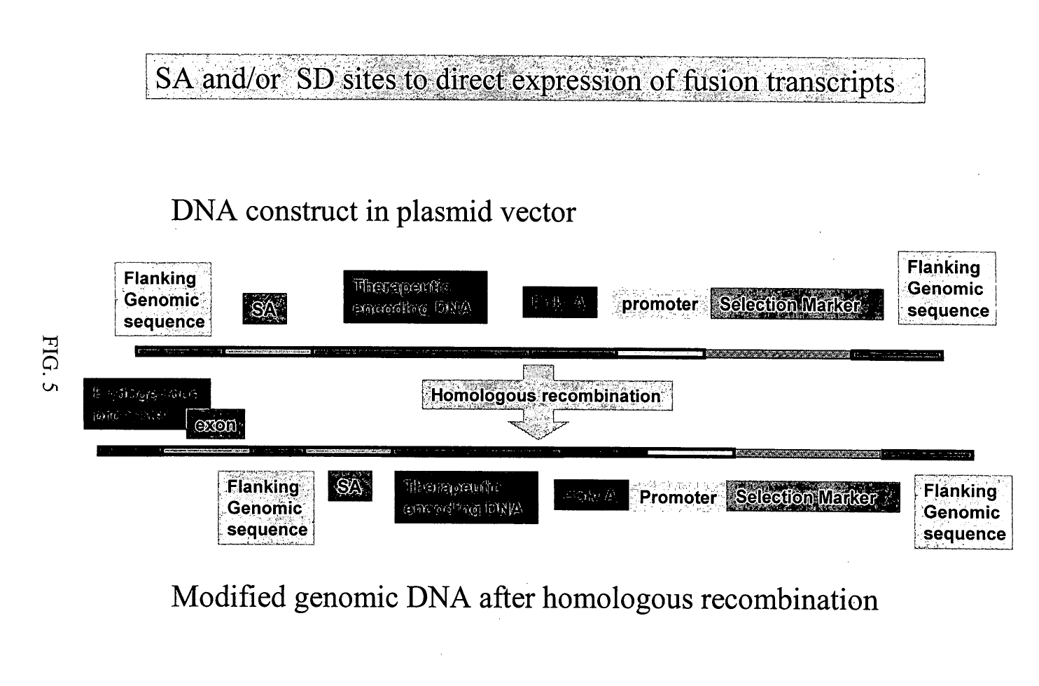

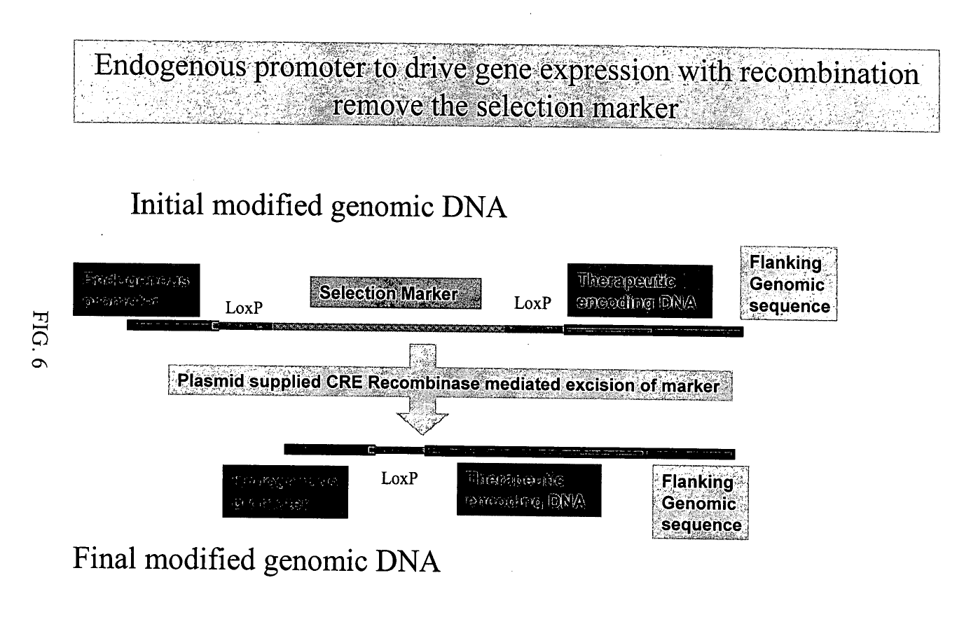

A vector may be a construct with either a splice donor or splice acceptor site to allow expression following integration into specific regions of the targeted locus. A vector may be a construct with an IRES site to allow efficient expression of the desired protein following integration into a specific region of the endogenous gene. Further, according to the present invention, a suitable vector may be any variation of such constructs. Examples of such recombination are shown in FIGS. 3-6.

### EXAMPLE III

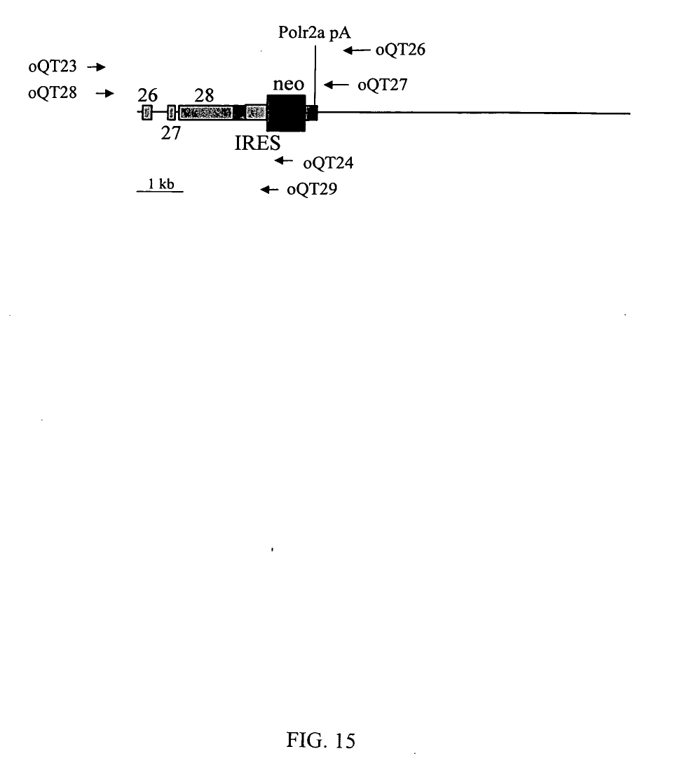

A vector was designed for homologous recombination. To construct a sequence that targeted the mouse Polr2a locus, IRES-neo sequences were cloned into the 3′ non-coding sequence (flanking exon 28) of the mouse Polr2a locus. (FIG. 15) (SEQ ID. NO: 1). Specifically, we inserted an internal ribosomal entry site (IRES) element linked to the gene for neomycin resistance (neo) in a genomic DNA fragment containing the last three exons and the 3′ untranslated region (3′UTR) of the Polr2a gene (FIG. 3; 3′UTR is depicted as a hatched box, pA is polyadenylation signal). The neo gene lacks any promoter sequence; it is translated from a second cistron using the IRES element and its expression is dependent on proper integration in the genome, i.e. 3′ of the endogenous promoter. This strategy greatly enhances the frequency of homologous recombination at a given locus (Tvrdik and Capecchi, unpublished observation). We chose the Polr2a gene, encoding the large subunit of RNA polymerase II, because it is an essential gene with high enough expression to ensure sufficient levels of neomycin resistance. The final targeting vector was linearized and introduced in GRP cells using electroporation (Expt 4a and Expt 4b) or lipofection (Expt 4c). The cells were allowed to recover for 24 hours and then placed in medium containing 70 micrograms/ml G418. In Experiment 1, 10⁸ GRPs were electroporated. In Experiment 2a and 2b, 2×10⁷ GRPs were used.

### EXAMPLE IV

Targeted transgene integration via homologous recombination in mammalian somatic stem cells was performed (Experiment 4a, Experiment 4b and Experiment 4c) that targeted transgene integration to specific sequences in the 3′ untranslated sequence of the Polr2a gene of glial progenitor stem cells (GRPs) isolated from embryonic mouse brain.

Procedures for isolating and culturing mouse GRPs have been previously published. GRPs, expanded by thawing and passaging of frozen primary cells, were cultured in DMEM/F12, 1×N2 supplement, 1×B27 supplement, 20 ng/ml of human basic FGF and 1× penicillin and streptomycin. In Experiment 4b and 4c, B27 supplement lacking retinoic acid was used. Cells could be efficiently transfected by either electroporation (˜40% of surviving cells transiently expresses a reporter gene) or Lipofection™ using Fugen Transfection Reagent (˜12% of cells transiently expressed a reporter).

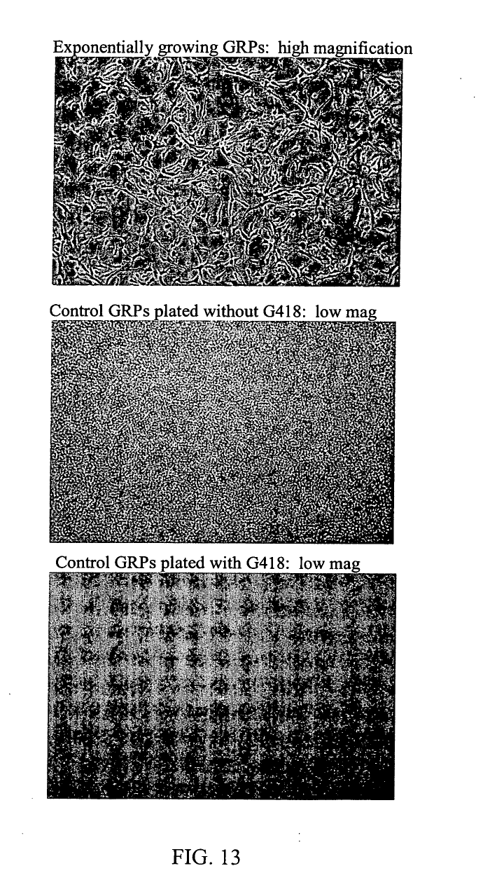

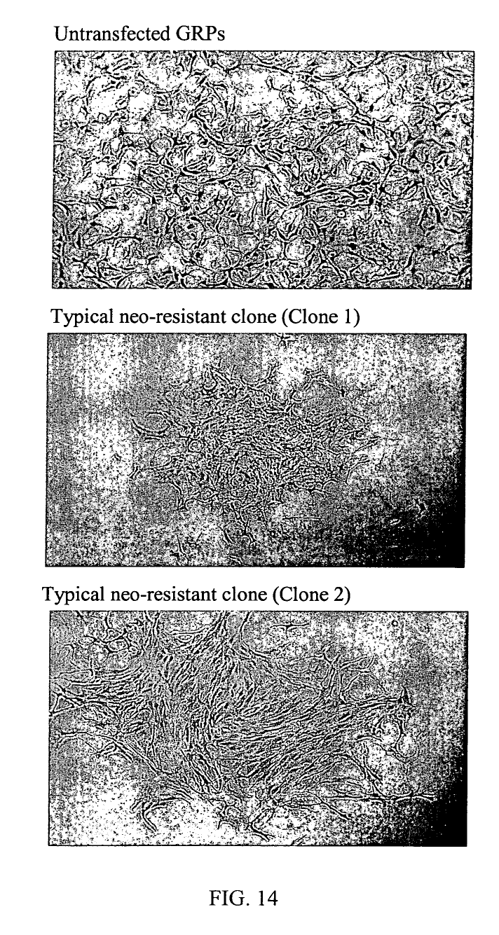

Primary GRP cultures are sensitive to neomycin (FIG. 13) and thus, selection for resistance to G-418 following cell transfection allows isolation of cell clones expressing a stably integrated neomycin resistance marker (FIG. 14).

Cells were transfected with the vector of Example III using electroporation (Experiment 4a and Experiment 4b) or Lipofection™ (Experiment 4c), allowed to recover for 24 hours and then placed in 70 micrograms/ml G418. In Experiment 4a, 10(8) GRPs were electroporated. In Experiment 4b and Experiment 4c, 2×10(7) GRPs were used.

Neomycin positive clones were observed in all three independent transfection experiments (FIG. 14 shows examples from Experiment 4a). 57 clones were seen in Experiment 4a, 29 in Experiment 4b and approximately 100 in Experiment 4c (in which case the cell clones were often too close proximity to be easily distinguished). Cells from isolated clones were picked used to seed two tissue culture wells: one to be frozen, the other to be used to analyze the nature of IRES-neo sequence integration in the specific clone.

In Experiment 4a, nine clones grew to levels sufficient for molecular analysis by PCR using oligonucleotides shown in FIG. 15. This low success in growing the clones was attributed to the presence of retinoic acid in B27 supplement. In Experiments 4b and 4c, where B27 supplement lacking retinoic acid was used, 40 of 53 clones grew vigorously after being picked.

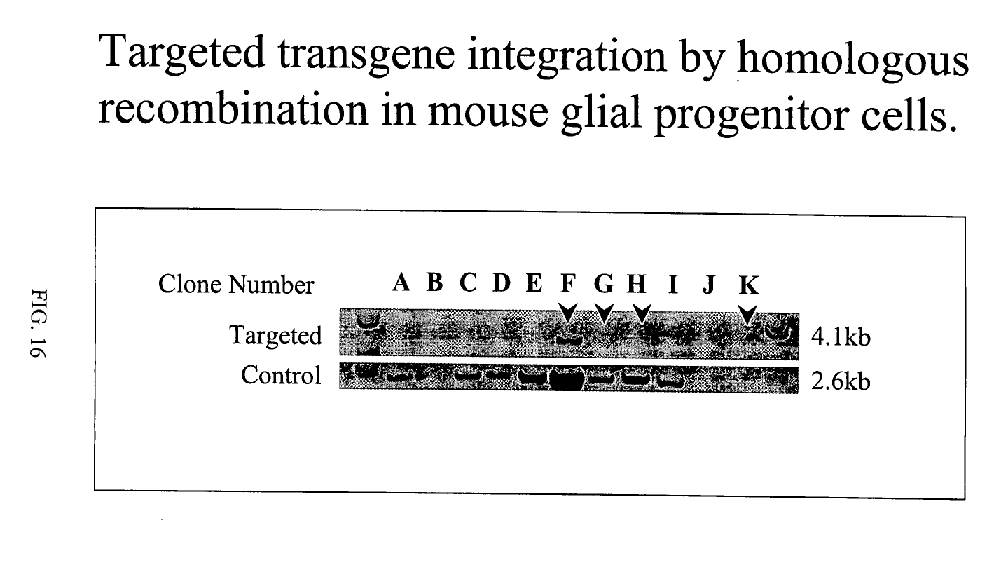

In FIG. 16, the PCR reaction was performed with oligonucleotides corresponding to Polr2a sequences, one contained in the targeting vector (QT26) and the other about 2.6 away in Polr 2a (QT23). In cases of homologous recombination, a 2.6 kb PCR fragment seen the wild type Polr2 locus, was expected to be interrupted by the 1.5 kb IRES-neo sequence and thus yield a ˜4.1 kb fragment.

In Experiment 4a, DNA from II clones (A-K) was prepared of which two (B and J) were discarded from consideration on the basis of the absence of a control band indicating sufficient genomic DNA for successful PCR analysis. All of the nine remaining clones showed presence of at least one wild-type Polr2a allele, as evidenced by the 2.6 kb PCR amplified fragment. However, four (F, G, H and K) showed an additionally 4.1 kb band, predicted to arise following homologous recombination mediated, targeted integration of IRES-neo into the 2.6 kb Polr2a fragment.

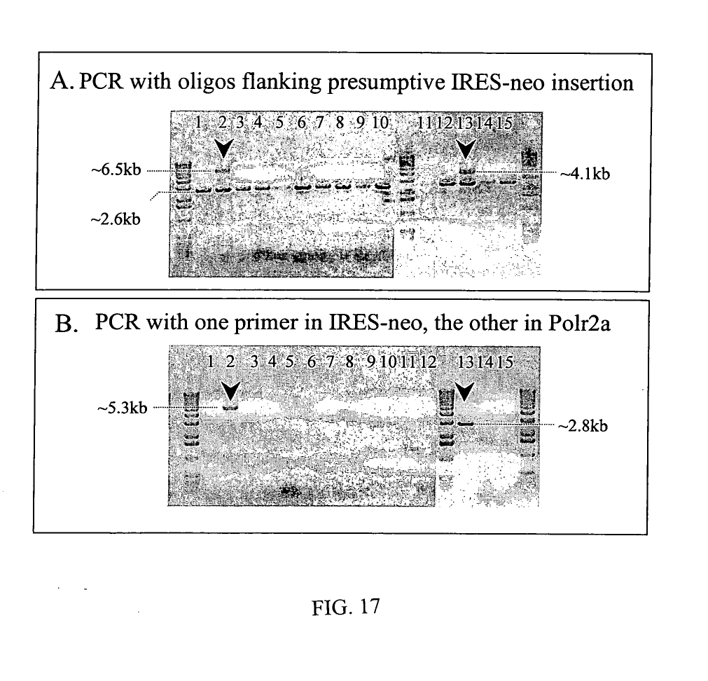

Two of 17 clones analyzed from Experiments 4b and 4c had IRES-neo integration into the Polr2a locus; one of these two resulted from homologous recombination (FIG. 17). In Experiments 4b and 4c, DNA from 15 clones (pooled results from 4b and 4c) were prepared and analyzed by PCR. In this analysis, two independent PCR amplification reactions were performed. First, as for Experiment 4a, an oligonucleotide pair flanking the presumptive IRES neo integration in Polr2a was used. One of the oligonucleotide sequences (QT26) was contained in the original targeting vector and the other (QT23) in Polr2 sequences not included in the vector. In all 15 clones analyzed, a control 2.6 band deriving from a wild-type Polr2a allele was observed (FIG. 17, part A). In two of the clones (2, 13) an additional larger band was observed. For clone 13, the band was ˜4.1 kb, precisely as predicted following homologous recombination-mediated targeted integration. For clone 2, the band is larger than 6 kb. Thus, while clone 2 carries a disrupted Polr2a gene, it is not likely to have arisen following a single homologous recombination event.

The involvement of additional rearrangements in clone 2 but not clone 13 is further evidenced by a second PCR analysis. In the second PCR analysis (FIG. 17, part B), the oligonucleotide pair was chosen such that a PCR amplified fragments should only be seen with template DNA from clones in which targeted integration has occurred. Thus, one of the oligonucleotide primers lies within the IRES itself, the other corresponds to Polr2a sequences flanking, but not included in, the targeting vector. A 2.8 kb band is predicted if IRES-neo was precisely integrated via homologous recombination. This 2.8 kb and is clearly visible in clone 13. In clone 2, a fragment was amplified indicating integration of IRES-neo in Polr2a, however this fragment significantly larger. This analysis provides additional evidence that in clone 2 (but not clone 13), IRES-neo integration into Polr2a was accompanied by additional rearrangements of local DNA. No fragments are amplified within any of the other 13 clones, consistent with data in FIG. 17, part A, indicating that IRES-neo integration in these clones occurred by a non-targeted mechanism.

Sequencing the IRES-Polr2a amplified fragment from clone 13 (FIG. 17, part B) confirmed, that the fragment derives from the Polr2a locus and confirms interpretation of the PCR data. The Polr2a locus is not expected to be unique in allowing a relatively high frequency of homologous recombination as ˜200 loci in embryonic stem cells have reported comparable rates for most genes that are not transcriptionally silent.

### EXAMPLE V

Thus, the feasibility of successful homologous recombination in somatic stem cells, specifically in murine glial progenitor cells has been demonstrated. This technology is easily generalized to glial stem cells, as well other classes of somatic stem cells, in all mammals including Homo sapiens. In view of the results of Examples I, II, III, and IV, methods of maintaining and culturing stem cells are optimized such that stem and precursor cells express high levels of telomerase (TERT) synthesize TERT (an enzyme which repairs the tips of chromosomes which would otherwise shorten each time a cell divides) and are maintained in an undifferentiated state for at least ten generations, it is possible to obtain homologous recombination in other progenitor cell populations. To test this hypothesis, mesenchymal stem cells and astrocyte precursor cells are used and it is shown that homologous recombination is possible in these cell types.

### EXAMPLE VI

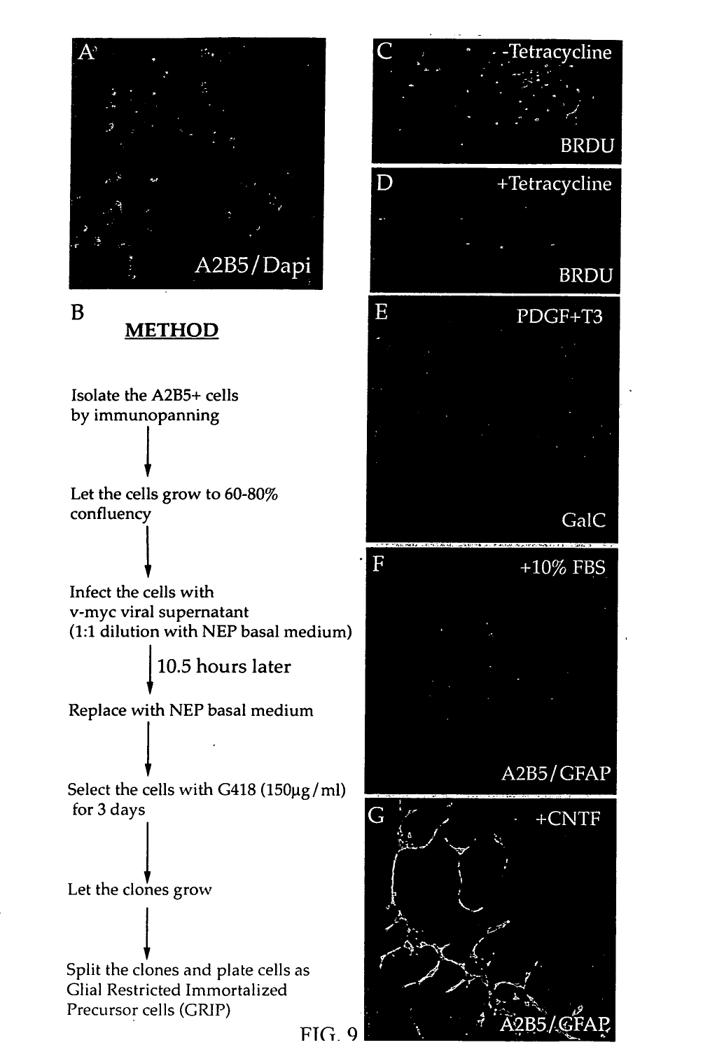

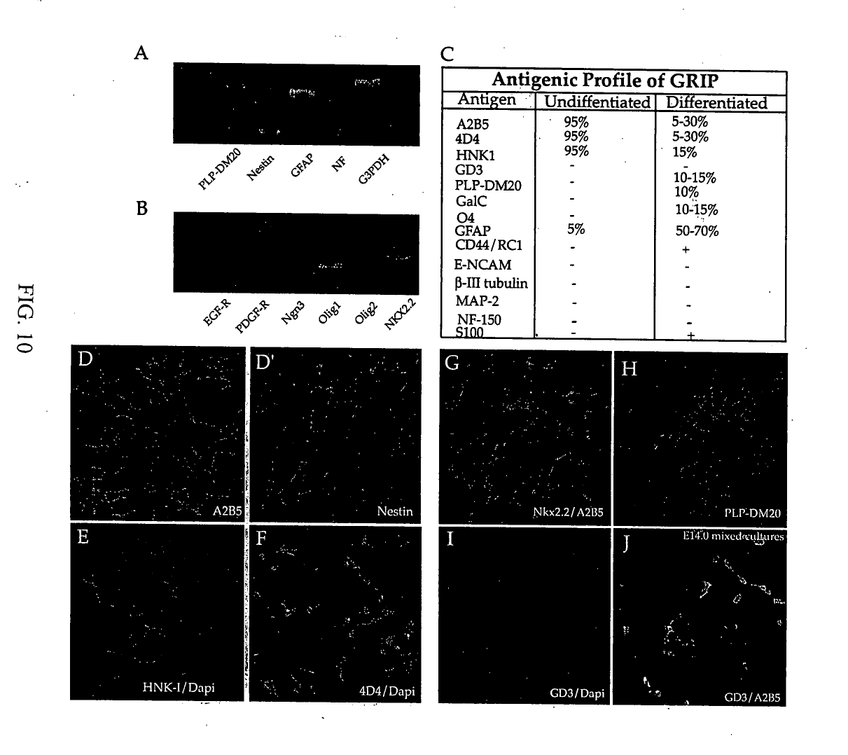

Foreign DNA may be inserted into cells and the cells may then be selected on that basis. Further, the insertion of foreign DNA does not alter the overall properties of the modified cells. (FIG. 9, FIG. 10; Wu et al. “Isolation of a Glial-Restricted Tripotential Cell Line from Embryonic Spinal Cord Cultures”; GLIA 38:65-79 (2002).) Stem or progenitor cells having DNA inserted into a homologous site are isolated and selected using a selectable gene marker. The cells are then used for subsequent experiments including, but not limited to, transplanting the stem or progenitor cells into a subject such that replacement of a gene product corrects an abnormality or deficit. Examples of such abnormalities include loss of a catalytic enzyme, reduction in levels of growth factors or their receptors and novel expression of a protein in a cell not normally expressing the protein. In the present invention, neo is expressed in glial progenitor cells at the Polr2a locus.

### EXAMPLE VII

In a related experiment, DNA encoding a therapeutic analgesic peptide is integrated into the Rosa locus of glial progenitor cells via homologous recombination. The glial progenitor cells are screened per the protocol of Example VI and transplanted in the spines of subjects, such as rodents. The glial progenitor cells secrete the integrated protein and are tested for efficacy in a rodent pain model.

### EXAMPLE VIII

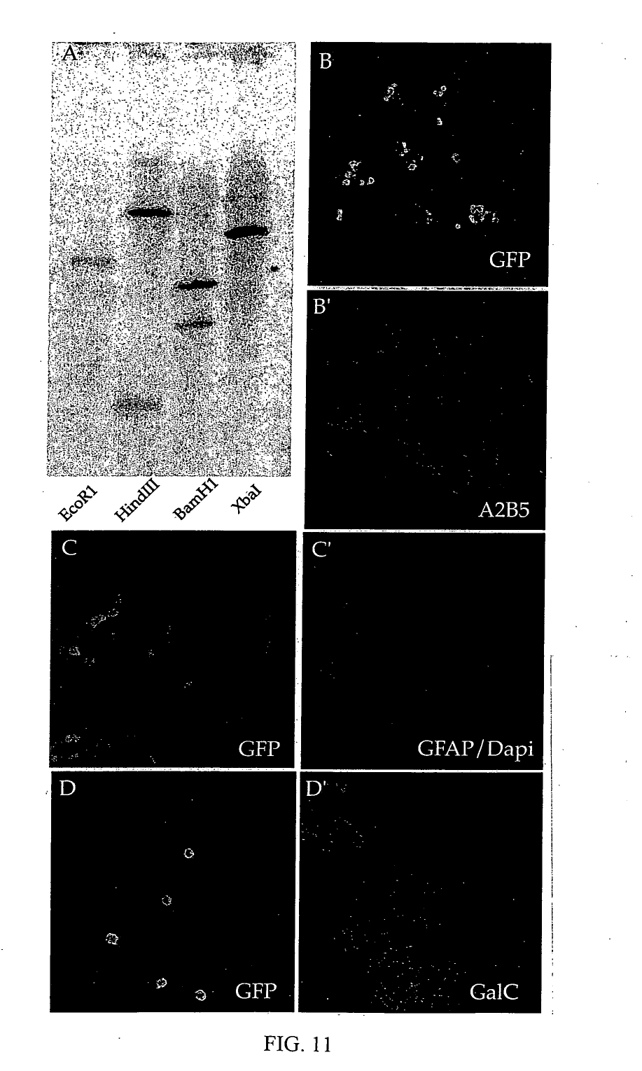

Cells may be retargeted for gene insertion to develop additional subclones. (FIG. 11; Wu et al. “Isolation of a Glial-Restricted Tripotential Cell Line from Embryonic Spinal Cord Cultures”; GLIA 38:65-79 (2002).) Progenitor cell lines in which at least one homologous recombination event successfully occurred are generated such that at least one exogenous sequence is placed in a selected site in the genome of a glial progenitor cell such that the same selected site is repeatedly targeted. For-example, an inserted gene sequence is replaced with a third gene or fourth gene in a reproducible manner.

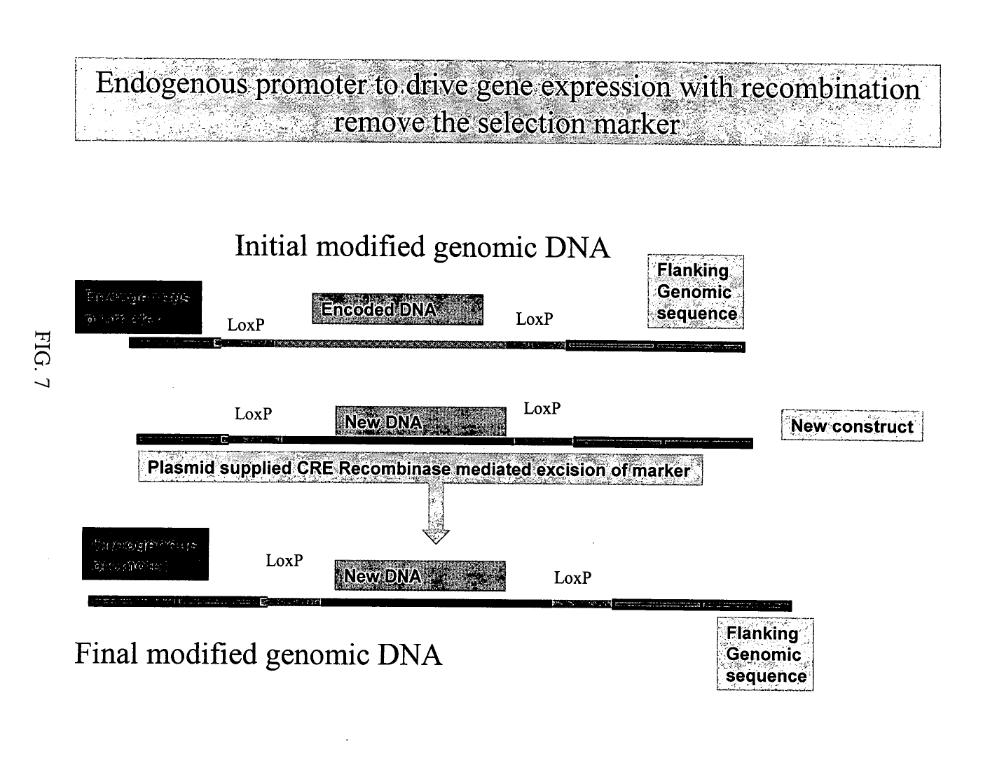

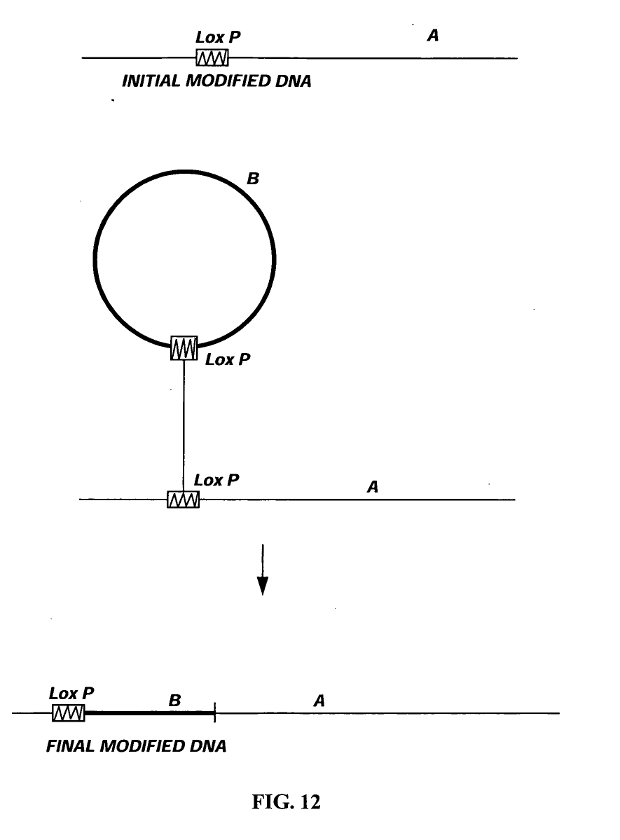

Once a site is specifically targeted and successful recombination is obtained, it is possible to retarget the same site at a substantially higher efficiency. One way to accomplish this is by engineering the BAC (bacterial artificial chromosome) including the locus of interest to contain alternative sequences at the targeted site (using homologous recombination in bacteria) and then using these new BACs as for performing homologous recombination in glial progenitor cells. A second way is to use a “floxed gene” (Cre/lox system), and other systems including ΦC31/AttP/AttB or Flγ/FRT, such that recombination occurs at the floxed locus at high efficiency replacing the existing locus with a new DNA. New DNA at the targeted site may serve to introduce a single site mutation, replace an existing exon or the entire gene. The new DNA may replace an existing sequence or may add to the existing sequence. A figure of one such strategy is shown in FIG. 7. Note, repeat targeting can be performed in several ways and one example using Floxed sites is shown. Another example of repeated targeting is shown in FIG. 12 wherein a single flox site is used to add a new DNA sequence. The techniques illustrated in FIG. 7 and FIG. 12 may be used in parallel or separately.

FIG. 4 depicts an example of using a vector containing an IRES site to direct expression of a transcript from an endogenous promoter.

### EXAMPLE IX

Homologous recombination is performed in a glial progenitor cell and multiple clones of the cell are obtained that express different candidate growth factors for evaluating the efficacy of growth factor delivery in vivo and allowing direct comparisons of gene expression. Thus, the glial progenitor cells act as delivery vehicles for the expressed proteins expressed by the genes. This process is also repeated for mesenchymal stem cells and astrocyte precursor cells.

The candidate factors include PDGF, a growth factor that triggers glial progenitor division and differentiation, and thus has potential for treatment of glial loss disorders including MS, ALS and leukodystrophies. Such factors also include GDNF, glutamate transporter and enzymes involved in leukodystrophies or lysosomal storage disorders. Another class of candidate therapeutic factor would cause increased secretion of therapeutic factors made by the glial cell: such molecules include dominant-negative forms of the mannose-6-phosphate receptors that, by inducing secretion of a large number of different lysosomal proenzymes, may generate cells useful for treatment of several different lysosomal storage disorders.

Glial progenitor cells are integrated with the gene encoding platelet-derived growth factor (“PDGF”) and introduced into the brain or spinal cord of a subject. The introduced cells express PDGF which promotes a proliferation of glial progenitor cells and their differentiation into oligodendrocytes. See, e.g., U.S. Pat. No. 4,889,919, U.S. Pat. No. 4,845,075, U.S. Pat. No. 4,766,073, U.S. Pat. No. 4,801,542, U.S. Pat. No. 4,350,687, U.S. Pat. No. 5,096,825, U.S. Pat. No. 5,439,818, U.S. Pat. No. 5,229,500, U.S. Pat. No. 6,077,829, U.S. Pat. No. 5,438,121, U.S. Pat. No. 5,180,820, U.S. Pat. No. 6,221,376, U.S. Pat. No. 6,093,802, U.S. Pat. No. 6,362,319 and U.S. Pat. No. 4,997,929, the contents of each of which are incorporated herein by reference.

Glial progenitor cells are integrated with the gene encoding epidermal growth factor (“EGF”) and introduced into the brain of a subject. The introduced cells express EGF which maintains neural stems cells in a proliferative state.

Glial progenitor cells are integrated with the gene encoding brain-derived neurotrophic factor (“BDNF”) and introduced into the brain of a subject. The introduced cells express BDNF which facilitates the survival and differentiation of neuronal precursors in the subventricular zone implicating a possible role in the treatment of Huntington's disease.

Glial progenitor cells are integrated with the gene encoding ciliary neurotrophic factor (“CNTF”) and introduced into the brain of a subject. The introduced cells express CNTF.

Glial progenitor cells are integrated with a cDNA encoding a lysosomal enzyme such as the tripeptidyl aminopeptidase-1 (TPP-1). The introduced cells overexpress and secrete TPP-1 of therapeutic benefit for many forms of LINCL/Batten's disease.

Glial progenitor cells are integrated with a cDNA encoding the soluble extracytoplasmic form of a mannose-6-phosphate receptor. The introduced cells secrete a large number of different lysosomal proenzymes at high levels and may be useful for treating nervous system defects associated with varied lysosomal disorders.

### EXAMPLE X

Homologous recombination is performed at a first locus in glial progenitor cells and then the obtained clone is reselected for a second recombination event which duplicates the change introduced by the first recombination event at the second allele. Such homozygous mutant cells may be obtained by either reselecting using a higher concentration of the selection agent or undertaking a second recombination process as the first in the same cell line.

Homologous recombination in cultured cells will generally target one allele of the locus of interest. To obtain cell lines homozygous at this locus one of two strategies can be attempted. Growth in high concentration of the selection agent can be used to obtain homozygotes or the site can be retargeted in a second recombination event as described earlier.

Although described with the aid of various illustrative embodiments and examples, the invention is not necessarily so limited.

TABLE 1
Stem cells present in selected tissues. Only a partial list has been
compiled to illustrate that tissue-specific stem cells have been isolated
from all three major germ layers and selected organ systems.

| | Cells | Properties |
|---|---|---|
| Ectoderm | Neural stem cell | Self-renewing and able to differentiate into |
| | | neurons, astrocytes and oligodendrocytes |
| | Neural crest stem | Self-renewing and able to generate neurons and |
| | cell | Schwann cells |
| | Skin-derived stem | Able to generate neural, glia, smooth muscle cells |
| | cell | and adipocytes |
| Mesoderm | Muscle-derived stem | Multipotent and self-renewal. Not committed to |
| | cells | myogenic lineage only |
| | Circulating skeletal | Multipotent with both osteogenic and adipogenic |
| | stem cells | potential |
| | Processed | Differentiate into adipogenic, chondrogenic, |
| | lipoaspirate | myogenic, and osteogenic cells |
| | Mesenchymal stem | Give progenies committed to a specific phenotypic |
| | cell | pathway in cartilage or bone tissue |
| | Umbilical cord blood | Self-renewing and multipotent |
| | stem cell | |
| | Hematopoietic stem | Self-renewing and multipotent |
| | cell | |
| Endoderm | Fetal liver epithelial | Form hepatocytic cluster and generate parenchymal |
| | progenitor cells | and bile duct cells |
| | Facultative liver stem | Bone marrow origin, generate epithelial cells |
| | cells (oval cells) | within the liver, hepatocytes and bile ductular cells |
| | Embryonic renal | Differentiate into myofibroblasts, smooth muscle, |
| | epithelial stem cells | and endothelial cells |
| | Pancreatic islet stem | Differentiate ex vivo into pancreatic endocrine, |
| | cells | exocrine, and hepatic phenotype |
| | Intestinal epithelial | Give rise predominantly to enterocytes, |
| | stem cell | mucus-secreting Goblet cells, peptide hormone |
| | | secreting enteroendocrine cells, Paneth cells and M |
| | | cells |

TABLE 2
Precursor cells present in selected tissues. Only a partial list
has been compiled to illustrate that precursor cells have been
isolated from all three major germ layers and selected organ
systems. Note more than one kind of progenitor cell is usually
present in any organ. References included serve as an
example and are not meant to be comprehensive.

| | Cells | Reference |
|---|---|---|
| Ectoderm | Skin-derived mast cell | (Kambe et al., 2001) |
| | Schwann cell precursor | (Jessen et al., 1994) |
| | Keratinocyte transient amplifying | (Lehrer et al., 1998) |
| | cells | |
| | Melanocyte precursor cells | (Silver et al., 1969) |
| Mesoderm | Ductular progenitor cell | (Sell, 2001) |
| | Hematopoietic progenitor cells | (Metcalf, 1998) |
| | Metanephrogenic mesenchyme | (Al-Awqati and |
| | precursor cells | Oliver, 2002) |
| | Adipocyte precursors | (Van and Roncari, |
| | | 1982) |
| | Muscle precursor cells (myoblasts) | (Yiou et al., 2002) |
| | Chondrocyte precursor cells | (Fang and Hall, 1997) |
| | Osteoprogenitor cells | (Long et al., 1995) |
| | Fetal lung mesenchyme cells | (Akeson et al., 2000) |
| | Thymus-derived myoid precursor cell | (Oka et al., 2000) |
| Endoderm | Pancreas precursor cells | (Alpert et al., 1998) |
| | Ureteric bud precursor cells | (Al-Awqati and |
| | | Oliver, 2002) |

### References

1. U.S. Pat. No. 4,350,687, issued Sep. 21, 1982 to Lipton et al.

2. U.S. Pat. No. 4,766,073, issued Aug. 23, 1988 to Murray et al.

3. U.S. Pat. No. 4,801,542, issued Jan. 31, 1989 to Murray et al.

4. U.S. Pat. No. 4,845,075, issued Jul. 04, 1989 to Murray et al.

5. U.S. Pat. No. 4,889,919, issued Dec. 26, 1989 to Murray et al.

6. U.S. Pat. No. 4,997,929, issued Mar. 05, 1991 to Collins et al.

7. U.S. Pat. No. 5,096,825, issued Mar. 17, 1992 to Barr et al.

8. U.S. Pat. No. 5,180,820, issued Jan. 19, 1993 to Barde et al.

9. U.S. Pat. No. 5,229,500, issued Jul. 20, 1993 to Barde et al.

10. U.S. Pat. No. 5,413,923, issued May 9, 1995 to Kucherlapati et al.

11. U.S. Pat. No. 5,416,260, issued May 16, 1995 to Koller et al.

12. U.S. Pat. No. 5,439,818, issued Aug. 08, 1995 to Fiddes et al.

13. U.S. Pat. No. 5,438,121, issued Aug. 01, 1995 to Barde et al.

14. U.S. Pat. No. 5,464,764, issued Nov. 07, 1995 to Capecchi et al.

15. U.S. Pat. No. 5,487,992, issued Jan. 30, 1996 to Capecchi et al.

16. U.S. Pat. No. 5,574,205, issued Nov. 12, 1996 to Kucherlapati et al.

17. U.S. Pat. No. 5,627,059, issued May 06, 1997 to Capecchi et al.

18. U.S. Pat. No. 5,631,153, issued May 20, 1997 to Capecchi et al.

19. U.S. Pat. No. 6,077,829, issued Jun. 20, 2000 to Tanaka et al.

20. U.S. Pat. No. 6,093,802, issued Jul. 25, 2000 to Lin et al.

21. U.S. Pat. No. 6,139,835, issued Oct. 31, 2000 to Kucherlapati et al.

22. U.S. Pat. No. 6,204,061 B1, issued Mar. 20, 2001 to Capecchi et al.

23. U.S. Pat. No. 6,221,376 B1, issued Apr. 24, 2001 to Lin et al.

24. U.S. Pat. No. 6,362,319 B1, issued Mar. 26, 2002 to Lin et al.

25. ARBONES et al., “Gene Targeting in Normal Somatic Cells: Inactivation of the Interferon-γ Receptor in Myoblasts,” Nature Genetics, Vol. 6, January 1994, pp. 90-159).

26. BUNZ et al., “Targeted Inactivation of p53 in Human Cells Does Not Result in Aneuploidy,” Cancer Research 62, Feb. 15, 2002, pp. 1129-1133.

27. CONG et al., “Human Telomerase and Its Regulation,” Microbiol. Mol. Biol. Rev., September 2002, pp. 407-425.

28. COPELAND et al., “Recombineering: A Powerful New Tool for Mouse Functional Genomics,” Nature Reviews/Genetics, Vol. 2, October 2001, pp. 769-779.

29. COURT et al., “Genetic Engineering Using Homologous Recombination,” Annu. Rev. Gener. 2002, 36:361-88.

30. HANSON et al., “Analysis of Biological Selections for High-Efficiency Gene Targeting,” Mol. Cell. Biol., January 1995, pp. 45-51.

31. HANSON et al., “Effects of c-myc Expression on Cell Cycle Progression,” Mol. Cell. Biol., September 1994, pp. 5748-5755.

32. PROUTY et al., “A Cell Culture Model System for Genetic Analyses of the Cell Cycle by Targeted Homologous Recombination,” Oncogene (1993) 8, pp. 899-907.

33. RAO et al., “A Tripotential Glial Precursor Cell is Present in the Developing Spinal Cord,” Proc. Natl. Acad. Sci. USA, Vol. 95:3996-4001, March 1998.

34. RAO et al., “Glial-Restricted Precursors Are Derived from Multipotent Neuroepithelial Stem Cells,” Developmental Biology, 188:48-63, 1997.

35. SEDIVY, John M., “Can Ends Justify the Means?: Telomeres and the Mechanisms of Replicative Senescence and Immortalization in Mammalian Cells,” Proc. Nat. Acad. Sci. USA, Vol. 95:9078-9081, August 1998.

36. SOMMER et al., “Neural Stem Cells and Regulation of Cell Number,” Progress in Neurobiology, 66:1-18 (2002).

37. WU et al., “Isolation of a Glial-Restricted Tripotential Cell Line From Embryonic Spinal Cord Cultures,” GLIA 38:65-79 (2002).

38. YANEZ et al., “Differential Effects of Rad52p Overexpression on Gene Targeting and Extrachromosomal Homologous Recombination in a Human Cell Line,” Nucleic Acids Research, 2002, Vol. 30, No. 3, pp. 740-748.

39. YANEZ et al., “Influence of DNA Delivery Method on Gene Targeting Frequencies in Human Cells,” Somatic Cell and Molecular Genetics, Vol. 25, No. 1, 1999, pp. 27-31.

40. YANEZ et al., “Therapeutic Gene Targeting,” Gene Therapy, 1998, 5, pp. 149-1 59.

41. YANEZ et al., “Uracil Incorporation Into a Gene Targeting Construct Reduces the Frequency of Homologous and Nonhomologous Recombinants in Human Cells,” Mutation Research 461, 2000, pp. 157-162.

What is claimed is:

1. A method of obtaining homologous recombination in somatic stem or progenitor cells, the method comprising:
growing stem or progenitor cells in culture;
inserting a nucleic acid encoding a gene of interest into the somatic stem or progenitor cells;
allowing homologous recombination to occur to produce a homologously recombined stem or progenitor cell; and
selecting a homologously recombined somatic stem or progenitor cell having the inserted nucleic acid.

2. The method according to claim 1, further comprising identifying somatic stem or progenitor cells that remain undifferentiated, express TERT, maintain telomerase activity, and demonstrate a capacity of self-renewal for insertion of the nucleic acid encoding the at least one gene of interest.

3. The method according to claim 1, further comprising identifying homologously recombined stem or progenitor cells producing a product encoded by-the at least one gene of interest.

4. The method according to claim 3, further comprising associating the homologously recombined stem or progenitor cell with a pharmaceutically acceptable carrier.

5. The method according to claim 1, further comprising introducing said homologously recombined stem or progenitor cell to a subject.

6. The method according to claim 5, wherein said introducing comprises in vitro delivery.

7. The method according to claim 5, wherein said introducing comprises in vivo delivery.

8. The method according to claim 5, further comprising selecting a subject incapable of producing a product encoded by the at least one gene of interest.

9. The method according to claim 8, wherein the product is a protein.

10. The method according to claim 5, further comprising selecting a subject incapable of expressing normal levels of a product encoded by the at least one gene of interest.

11. The method according to claim 4, further comprising introducing the homologously recombined stem or progenitor cell and the pharmaceutically acceptable carrier to a subject.

12. The method according to claim 1, further comprising providing a selection medium comprising growth medium for the homologously recombined somatic stem or progenitor cell, the growth medium including a selection agent.

13. The method according to claim 1, further comprising selecting the somatic stem or progenitor cells from the group consisting of glial progenitor cells, mesenchymal stem cells, astrocyte precursor cells, and mixtures thereof.

14. The method according to claim 1, wherein the somatic stem or progenitor cells are glial progenitor cells.

15. The method according to claim 1, wherein inserting nucleic acid into the somatic stem or progenitor cells comprises using a vector capable of homologous recombination.

16. The method according to claim 15, wherein the vector comprises regions of homology with DNA of the stem or progenitor cells.

17. The method according to claim 16, wherein the regions of homology are selected from the group consisting of Rosa locus, RNApoII locus and the beta-actin locus.

18. The method according to claim 17, wherein the regions of homology are from the RNA polr2a locus.

19. The method according to claim 1, further comprising inserting the nucleic acid by a method selected from the group consisting of electroporation, lipofection, cell fusion, retroviral infection, cationic agent transfer, CaPO₄, transfection and combinations thereof.

20. The method according to claim 19, wherein the method is electroporation.

21. The method according to claim 1, further comprising introducing an IRES protein at a locus of nucleic acid of the somatic stem or progenitor cells prior to inserting the nucleic acid into the somatic stem or progenitor cells.

22. The method according to claim 1, further comprising identifying a promoter in the nucleic acid and modifying the promoter to alter expression of a product encoded by the at least one gene of interest.

23. The method according to claim 22, further comprising replacing at least a portion of the promoter with a product capable of providing additional regulation of expression of the product encoded by the at least one gene of interest.

24. The method according to claim 5, wherein introducing comprises introducing the homologously recombined stem or progenitor cells to the brain of the subject.

25. The method according to claim 5, wherein introducing comprises introducing the homologously recombined stem or progenitor cells to the spinal cord of the subject.

26. The method according to claim 1, wherein the at least one gene of interest encodes at least one growth factor.

27. The method according to claim 26, wherein the at least one growth factor is selected from the group consisting of platelet derived growth factor, epidermal growth factor, fibroblast growth factor, brain derived neurotrophic growth factor, glial derived neurotrophic factor and ciliary neurotrophic factor.

28. The method according to claim 5, further comprising obtaining multiple homologously recombined stem or progenitor cells.

29. The method according to claim 28, further comprising introducing the multiple homologously recombined stem or progenitor cells to the subject.

30. The method according to claim 29, further comprising evaluating the efficacy of product delivery in vivo.

31. A homologously recombined stem or progenitor cell encoding a gene of interest capable of expressing a selected product.

32. The homologously recombined stem or progenitor cell of claim 31, wherein the homologously recombined stem or progenitor cell is capable of expressing an endogenous protein encoded by nucleic acid integrated in the somatic stem or progenitor cell via homologous recombination.

33. The homologously recombined stem or progenitor cell of claim 31, wherein the somatic stem or progenitor cell is selected from the group consisting of glial progenitor cells, mesenchymal stem cells or astrocyte precursor cells.

34. The homologously recombined stem or progenitor cell of claim 31, wherein the somatic stem or progenitor cell is a glial progenitor cell.

35. The homologously recombined stem or progenitor cell of claim 31, wherein the homologously recombined stem or progenitor cells are incapable of expressing MHC class antigens.

36. The homologously recombined stem or progenitor cell of claim 31, wherein the homologously recombined stem or progenitor cells are capable of differentiating.

37. The homologously recombined stem or progenitor cell of claim 31, wherein the homologously recombined stem or progenitor cells are capable of expressing TERT.

38. The homologously recombined stem or progenitor cell of claim 31, wherein the homologously recombined stem or progenitor cells are capable of maintaining telomerase activity.

39. The homologously recombined stem or progenitor cell of claim 31, wherein the stem or progenitor cells are capable of self renewal.

40. A method of gene therapy comprising administering to a subject a homologously recombined stem or progenitor cell such that the homologously recombined stem or progenitor cell express a gene product of interest.

41. The method of gene therapy of claim 40, wherein the homologously recombined stem or progenitor cell expresses an endogenous protein encoded by nucleic acid integrated in the stem or progenitor cell through homologous recombination.

42. The method of gene therapy of claim 40, further comprising selecting the homologously recombined somatic stem or progenitor cells from the group consisting of homologously recombined glial progenitor cells, homologously recombined astrocyte precursor cells and homologously recombined mesenchymal stem cells.

43. The method of gene therapy of claim 42, wherein the homologously recombined somatic stem or progenitor cells are homologously recombined glial progenitor cells.

44. The method of gene therapy of claim 40, wherein the homologously recombined stem or progenitor cell are adapted for used in treating neurological or neurodegenerative disorders.


SEQUENCE LISTING (1 sequence)

SEQ ID NO: 1 - DNA, 12607 bp, Mus musculus, synthetic
  misc_feature (1)..(2166): Region pUC vector
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgagttcccctttggcactgctttccactgttgattgttgggcaggatgggtgctgtgggtgatgggtgctgtgggcagggagattaatgtctcttctggttgttcaggtggatgtccttatggaagcagcagcacatggggagagtgacccgatgaagggagtctctgagaatattatgctgggccagctcgctccagctggtactggctgttttgacctcctgcttgatgctgagaagtgcaaatacggcatggaaatccccaccaatatccctggcctgggggctgctggacgtgagtgtgtgggctttcagctgtgtcaagctggctgggtcatgcttaagggtagacatacaagggagtgcgtgcctttagccccgagtgctgctcctcataaaccactctgggctctttgcagctaccggcatgttctttggctctgcacccagtccgatgggaggaatatctcctgcaatgacaccctggaaccagggtgcaactcccgcctatggtgcctggtccccgagtgttggtgagtagcctcgtccaggaagagaagcgtcggacttcgtgggtttggggctggggaagggggtgctagagctcctgctgacgcacctgttcttccttcagggagcgggatgaccccaggagcggccggcttctctcccagtgctgcatctgatgccagtggcttcagcccaggttactcccctgcatggtctcccacaccaggctctccgggctcccctggaccctcaagcccatacatcccctcaccaggtgagctgctacaccatttcctccttcctccatgtttgtatgccgggctcttcaccactatttccctttgtcttttcctataggtggtgctatgtctcccagctactcaccgacatcaccagcctatgagccacgctcccctgggggctatacaccccagagcccctcctactcccctacttcaccttcctactccccaacctctccatcttactctccaaccagtcccaactacagccctacctctcctagctactcgcccacctcccctagctactcgccaacctctccttcctactcccccacctctccaagctattccccaacctctcctagctactccccaacctctccaagctattctccaacatcacctagctattctccaacttctcccagctactcaccgacatctcctagctactccccaacttctcccagctactcaccaacttcaccaagctattctcccacctcccccagctactcaccgacatctcccagctactcaccaacttctccaagctactcaccaacttctccaagctactcacccaccagccctaactattctccaactagtcccaactataccccgacatcacccagctacagcccaacctcacccagttactcacctacaagtcccaactatacacccaccagccctaattacagcccaacctctccaagctattccccaacctcacccagttattcccccacctcaccaagctactccccctccagcccacgatatacaccacagtctccaacctacacaccaagctcaccaagctacagtcccagctcaccaagctacagccccacttcacccaagtacaccccaactagtccttcctacagtcccagctcaccagagtacaccccagcttctcccaaatactcacctacaagccctaaatattcacccacttctcccaagtattctcctaccagccccacttactcacctaccaccccaaaatattctccaacctccccgacatactcaccaacctctccagtctataccccgacctctcccaagtactccccaaccagccctacctactcgcccacttctcccaagtactcgcccaccagtcccacctactcacccacctctcccaagggctccacctactctcccacttctcctggctactcacccactagccccacctacagcctcaccagcccagccatcagcccagatgacagcgatgaggagaactgagcgaacagggcgaagagctggttagggtcagacaacctcggtggcctgtgtgtcacttccctcacctcacaggctgtgaccctaccctgggtcccttgtacataactccttgtgacaaaaccctctggaagttctggaccccatttttgataggcttttttgtcttgtcctctactcatgctgtcttggactcactgacccagctgcagatatcgcggccgcgaagttcctattctctagaaagtataggaacttcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggatccgccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcgaagttcctattctctagaaagtataggaacttcgtcgactgcagggttttgttaccctgccagtcccctgcctcactccaccatgaaggtctctgctttcctgtgacttgattgggatacaggcatttaactttggaaggctagtttggtgcctgctctgggtcatacacctgatctggaagaacaaagcttaagctgcctttgttattttttgaaattgaaataaagtttactaattttgaccaaaagtctgtatgtgtactgactgcttaattgaggctcagagaggcaaaaattccaagctcactgagccttcttgaaaaactacgggggctggtgagatggctgagcaggtaagagcactgactgctcttccaaaggtcatgagttcaaatcccagcaaccacgcggtggctcacagccatccataacgagatctgatgcccttttctggagtgtctgaagtcagctacagtgtacttacatgaaaggaaggaaggaaggaaggaaggaaagaaaagaaaactacagaagatgctagtgcagaagaagcaaaaataatttgcctaaatcctaccatcaggaagcatgtctttgtttttgtttgtttgtttgcggggaggggggcatgtttgttataaagaaatgcctgtgtagtcctggctgtttggaagtgtctatactgaccaggtagcctccaactcagatatgttctgcctctgcctcaggagtgcaaggacctaaccttaaaatatttttgttaggatttgtttctaaatgagggattttttggtgttttgttttacatatataagtgtgtaccatatgtatctagtgcccaatgatcagaagagggcgttgggtctcttgataattatgagccaccatgggggcattggaaaccaaatccatgttctctgacagaacaagtggggttggttggtttttagtttaagactagggtttctcagtatagccttggctaccctcagactccccagtgcagggattaaagcaccactaccaccagcccagaacacatgttggcctaattgctgaaccatctctccagcttcccagtccccattttgatatttttattctacacataggagaatacctgcctctatctctggaatgctgggattatagacacatgccaccaaacctagccttataacttaattttttagaaagatttttgccagtaggggtagcacacaaatttaccagcactccagaggcagaggaaggcagagttgtacctggcctacatagtaacttccaggacagccagggctacatagtgaaatcttgtctcaaaaaacaaagtttaattaagctagagacatgaattctttgttaagagtgctcagtacacttggcagaggacccaagttcaatttccagcatacaccaggcagtagtcacaactacccataagctccacctagcctccaacacagcctgcattcagtacacacacacatatataagttaaaggtcttttaaaaacaaaaacgggagttatggtgctggagagatagctcagggtaagagcagtgtattgctcttgaggggacctgggtccaggcacacacttggcacacatacatacatgaagacaagacactcagaagtaaatctataaagggggtcacctatttttattttctgtgggtattcatgtctcagtgcattgtggtgccaggcctggctttcacatgaatgctggagacagcgcaagtcctgatgcttgaggggcagacactaccaaaagaccaaaggaatcagctctctagctttccagtcacagctttgatttccgaagttacttcttgatgagaaatgtaactgactagaacaattcatctttgcctttggccttttttcccctattttgagacttactattttatgtgtatgaatgtttcccttgcataggagtgcatgtaccacgtgcatgccaagtgcccacggtccacagagatcagaaggtggtgttggatcccctggaactagagttaggaatagttatgagtcaccctgtgcgtgctatcaggccgtctggaagaacactagagcagtctgcatctctcatgccctataaccattttttaaaaattaaacttttaggctagagaggtgggttagtggttgaggaccacctacatggttcaattcccagtacctacatggtggctcacagacatctgtaaccacagttccaggggatccaacaccttcctctgacctcagagggtaccaaacaaacctgtgtgggacacaggcatacatacaaacaaaatacccatatacacaaaataaaaattcattcaggatttaaaaagtggaaatttgccgggcgtggtggtgcacgcctttaatcccagcacttgggaggcagaggcaggcggatttctgagttcgaggccagcctggtctacaaagtgagttttccaggacagccagggctatacagagaaaccctgtctcagggggggtgggggggagtggaaatttcaggctggagagatggttcagaggttaagagcactgtctgctcttccagaggtcctgagttcaattctcagcaagctcgtggtggtttataaccatctgtaatgggatctagtgccctcttctggcccacaggatacatgcaggaagaatgctgtatacataattctctctctctttctctctctctctctgtcgtgtgtgtgtgtgtgtgtgtgtgtgtgtgtgtgtgtgtgtgtgtgtgtgtgtgtgtgtgtgtgtttcaagacagggtttctctgtgctagccctggctgtcccggaactcactctgtagaccaggctagcctcgaactcagaaatctgcctgcctcccaagtgctgggattttaaaaagtggaactttcagcaagctactacacttatctaatctgcaacaagatcttattacacaggaacattcagttttatgaagccagccatagtggtttcagtctgtaattccagcatgtcggtggccaagcaccaggactgctactagttggagcccagcttggactacatcctgaaagtgagaaaactgagctacagagtgagactcccaggagatacagatacaggattctatgaaactttgaggtcagctatgttgccgacattggcctcatgcttggaatcttcttgcatgacaccctcaagcattagaatcatgagtgtcagcatgccaaactttcattactgattatagactttaatgactgagccatttctccagccccttattagttttattatattttatttggttttgggatgtgtgcgtgtatgtgcatgtgtgtgcacgcacacatgtaatagtgccaatggagttcaaaagacaacttgtgagagttggttctctcccaggggtcaaactcaggcttcagcctctgtagcagatgccttgagccactgagccatcttgctgcctgcccattttaaaaataattaactgctgggtatggtggtacaaagctttaatcctagaactctggacacaaagccaatcagaactctatgtctgagaccagcctgatcttcatagcaagttccactccagtcagagctatgtagtgaggccctgtctaaatataaataaatagcacaagagctgcagccatagcacccagccagaatagcatgatccagaagcccctcttccacaaggattggcagcagtgaacgggcactcagttcaaccaactggcacaccagatccacaaacgcaaggccgggaatccaaagtgtgccacaatgccccctcacccagtgtcaggtcccatcaggcccatagtgaggtgccctatgatgagataccacaccaaggtccaagatggcagggactccagcctggaagaactcagggtgactggtatccacgagaaagtgggatgcagcatcgtggcaccttcgtggacccaaggagaagcaggccagcatgcagagcagcacctctccaagctcatgctttcccccaggaaaccctctgctccaaagggagctagttccgctgaagaacttatattacccaccggcttacaggacctgggatgcccatctggaatgtctacaaaaagggaaaagccagagccgccccagaagaagaggagtgggagactttgaccagtcttagggtccgggccagtgcccggctccttgccacccaaatacaaagggcaaagaaagctggagaacaagatgccaaaaagtagtgagccattagagtattgcaataaaattcccataaggcaaataaataaacaaacagataaatacatgaaatagatggttgactgttactacgacatggctgaactttaattattttgttcttagaagcattgcttccctctttcctaccactatgaaaagttttttttttttttcatttaatagtttcattaatttgggcatgttatttagctccaaatgtttaaattgaatatttaaggaaattaaatggaaaaacggcctgtgatatagctcagccagtgaaggtgcctgctgacctgctgacctgatttccatccctgggagcccacatggtacaagggaggaccaactcctataaattgtcttgtcctctctctctctctctctctctctctctctctctcatgtgtgtgtgtgtgtgataaagatgactctgtacttaagagcacatgtgctgctcttccagaagacctgagttcagctcccagcatctgtgtcagaagttcataatcactctagctccaggctgtctgatgcctctaaaaaaatacctgcattcaaatgcacaaacccacactcaggcacccacatacacacaaataaatatttttgatgcagtaaaggagttgttaagagcacttgctgttcttgcagaagactcaggtttggttcctggcactcccatggtggtttcttgtccttgactccaattcaggggatatggcgccctcttctggcaactgaacgtaccaagaatgcactcagcgcacttacatactggcagaaaatatactcatacacataaaaactctaaatcttggggctagagagatggctcagtggttaggagcccctggatctggctggtcttatagaggatccagggttcaattcccagcaccaagtggcagctggcaactgtaactccagttccaggagatctgacaccctcatagaaacacacacaggaaaatgtgtataaaaatataattttaagagccaggaagtgatggcacacacctttaattccagcacttgggaggcagaggcaggtggatttctgagtttgaggccaacctggtctatagagtgagttccaggacagccagggctacacagagagactctgtctcaaaaaaccaaaaataaataaataaaaataaaatgttaaaataataaaaaattattaataataataacaacaacaagaatagatgaagaacaacaacaacaacaacaatcatgggcatttatttttctagggtaactttcccaggtggaattgaattttctcattggagatgggtctcagaagcaacgttcccagtcctgtgcttaactaactataattttggaagaaattcaaaatctgaccaaaactacaaataaaagctaagtgtggtggttcatgcctctgaccccagctctcaggaggcagaggcaggcagatccctgtaagtttgagaccagcctggtcatccagggctacacagaaaaaccctatcttaaaaaacaaaatcaggggctggtgagatggctcaggggttaagagcgccgactgctcttcctaaggtcctgagttcaagtcccagcaaccacatggtggctcacaaccatcggtaatgagatctgactccctcttctggagtgtctgaagacagctacagtgtactcacatataataagtaaataaatattaaaaaaaaaaaaaaaccaaaatcaaagtctttaaagttcgataaaaaaaattaaagaagaaaatagtattgttttgtgtccccctagataggctgtgttgcccaattcacgtgctcctgagggcctgagacatgcgctcttttccttcagcataggaaactcccttttccatactccctgcccagcagtgttgcttgtggtagacgcttggtttttatctggagttctccattctcccttcaaagggatatctcccccgacctcagcgacgtcatgcatagcgctgcgatcgcgttaaccccgggtcgcgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctttgaccgtctccgggagctgcatgtgtcagaggttttcaccgtcatcaccgaaacgcgcgagacgaaagggcctcgtgatacgcctatttttataggttaatgtcatgataataatggtttcttagacgtcag